SEQUENCE LISTING (7 sequences)

SEQ ID NO: 1 - DNA, 60 bp, synthetic construct
  source 1..60: mol_type = other DNA
atgacgcgctttcaagcgtggcgagtatgtgaaccaaggcttcggacaggagatcggaag

SEQ ID NO: 2 - DNA, 50 bp, synthetic construct
  source 1..50: mol_type = other DNA
actatatacttaggtttgatctcgccccgagaactgtaaacctcaacatt

SEQ ID NO: 3 - DNA, 50 bp, synthetic construct
  source 1..50: mol_type = other DNA
tgaaatatcttaggtttgatctcgccccgagaactgtaaacctcaacatt

SEQ ID NO: 4 - DNA, 50 bp, synthetic construct
  source 1..50: mol_type = other DNA
ggcctcgcggaggcatgcgtcatgctagcgtgcggggtactcttgctatc

SEQ ID NO: 5 - DNA, 50 bp, synthetic construct
  source 1..50: mol_type = other DNA
gagaattattcgggggcagtgacaaccaacatctcgggtcctgcccaacc

SEQ ID NO: 6 - DNA, 50 bp, synthetic construct
  source 1..50: mol_type = other DNA
ggtctacacgctaatatagcgaatcaccgagaacccggcgccacgcaatg

SEQ ID NO: 7 - DNA, 50 bp, synthetic construct
  source 1..50: mol_type = other DNA
gaacgtccttaactccggcaggcaattaaagggaacgtatgtataacgca

(19) United States
(12) Patent Application Publication
(10) Pub. No.: US 2026/0152737 A1
(43) Pub. Date: Jun. 4, 2026

(54) SAMPLE PROCESSING TO ENRICH FOR SELECT SPECIES OF NUCLEIC ACIDS

(71) Applicant: Karius, Inc., Redwood City, CA (US)

(72) Inventors: Damek Spacek, Palo Alto, CA (US); Igor D. Vilfan, San Francisco, CA (US); Timothy A. Blauwkamp, Palo Alto, CA (US); Fred C. Christians, Redwood City, CA (US)

(21) Appl. No.: 19/345,645

(22) Filed: Sep. 30, 2025

Related U.S. Application Data
(63) Continuation of application No. PCT/US2024/025027, filed on Apr. 17, 2024.
(60) Provisional application No. 63/496,935, filed on Apr. 18, 2023.

(51) Int. Cl. C12N 15/10

(52) U.S. Cl. CPC C12N 15/1072; C12N 15/1065

(57) ABSTRACT

Disclosed herein are methods of preparing a nucleic acid library from a sample comprising a mixture of microbial nucleic acids and human nucleic acids, wherein the method comprises receiving the sample comprising a mixture of microbial nucleic acids and human nucleic acids, wherein one or more of the human nucleic acids comprise a 5′-end phosphate moiety, attaching a blocking moiety to the 5′-end phosphate moiety to the one or more human nucleic acids of the mixture or degrading the human nucleic acids using a 5′ phosphorylation-specific exonuclease, phosphorylating the microbial nucleic acids of the mixture to produce 5′ phosphorylated microbial nucleic acids, and attaching 5′-end adapters to the 5′ phosphorylated microbial nucleic acids to produce a nucleic acid library enriched for microbial nucleic acids.

### CROSS-REFERENCE

This application is a continuation application of International Patent Application No. PCT/US2024/025027, filed Apr. 17, 2024, which claims the benefit of U.S. Provisional Application Ser. No. 63/496,935, filed on Apr. 18, 2023, the entirety of which are hereby incorporated by reference herein.

### SEQUENCE LISTING

The instant application contains a Sequence Listing which has been submitted electronically in XML format and is hereby incorporated by reference in its entirety. Said XML copy, created on Apr. 16, 2024 is named 47697-743.601.xml and is 7,399 bytes in size.

### BACKGROUND

Next generation sequencing (NGS) can be used to gather massive amounts of data about the nucleic acid content of a sample. It can be particularly useful for analyzing nucleic acids in complex samples, such as clinical samples. However, before using NGS methods, a starting sample can often be obtained or extracted or subjected to multiple sample processing applications, which can reduce nucleic acid recovery, delay sequencing, delay reporting of clinical calls, introduce errors, introduce bias, and often results in chemical waste requiring controlled handling. Errors and biases can affect results in many cases, such as when there are low abundance nucleic acids or target nucleic acids in patient samples.

There is a need for more efficient and accurate methods for detecting and quantifying nucleic acids as well as for preparing nucleic acid libraries. This need is particularly pronounced when processing samples such as patient samples that may contain a low abundance of nucleic acids or target nucleic acids.

### SUMMARY

This Summary introduces a selection of concepts that are described further below in the Detailed Description. This Summary is not intended to limit the scope of the claimed subject matter.

In an aspect, the present disclosure provides a method of preparing a nucleic acid library from a sample, the method comprising: (a) providing a sample comprising a mixture of nucleic acids, wherein the mixture of nucleic acids comprises a first population of nucleic acids that are amenable to a first chemical reaction and a second population of nucleic acids that are not amenable to the first chemical reaction; and (b) selectively modifying the first population of nucleic acids such that the first population of nucleic acids is no longer amenable to the first chemical reaction; and (c) selectively modifying the second population of nucleic acids in the mixture such that the second population of nucleic acids becomes amenable to a second chemical reaction. In some embodiments, the second population of nucleic acids comprises a genotype that differs from the genotype of the first population of nucleic acids. In some embodiments, the second population of nucleic acids comprises microbial nucleic acids and the first population of nucleic acids comprises human nucleic acids. In some embodiments, the first or second population of nucleic acids comprises fetal nucleic acids, maternal nucleic acids, transplant recipient nucleic acids, transplant donor nucleic acids, mutant nucleic acids, or wild-type nucleic acids.

In some embodiments, the first and second populations of nucleic acids comprise DNA. In some embodiments, the first and second populations of nucleic acids comprise cell-free DNA.

In some embodiments, the first and second populations of nucleic acids comprise RNA. In some embodiments, the first population of nucleic acids comprises DNA and the second population of nucleic acids comprises DNA or RNA.

In some embodiments, the first chemical reaction in (a) is a ligation reaction. In some embodiments, the first and second chemical reaction are both ligation reactions.

In some embodiments, the method comprises in (a), 5′ termini of the first population of nucleic acids that are amenable to a chemical reaction are phosphorylated and in (a), 5′ termini of the second population of nucleic acids that are not amenable to the chemical reaction are not phosphorylated. In some embodiments, the second population comprises nucleic acids with natively unphosphorylated 5′ termini and the first population comprises nucleic acids with phosphorylated 5′ termini. In some embodiments, the second population comprises nucleic acids with a mixture of natively unphosphorylated 5′ termini or synthetically-phosphorylated 5′ termini and the first population comprises nucleic acids with natively phosphorylated 5′ termini, synthetically-phosphorylated 5′ termini or a mixture of natively phosphorylated 5′ termini and synthetically-phosphorylated 5′ termini. In some embodiments, the selectively modifying comprises modifying 5′ termini of the first population of nucleic acids to resist the first chemical reaction. In some embodiments, the first chemical reaction is a ligation reaction. In some embodiments, the method further comprises denaturing the sample.

In some embodiments, the selectively modifying comprises selectively attaching a blocking group to the 5′ termini of the first population of nucleic acids. In some embodiments, the blocking group comprises a 5′ terminus that is resistant to phosphorylation or ligation. In some embodiments, the blocking group comprises an identifying tag. In some embodiments, the blocking group is a splint oligonucleotide that comprises a double-stranded adapter region and a single-stranded region, wherein a splint region of the splint oligonucleotide comprises a first strand of the double-stranded adapter region connected to the single-stranded region. In some embodiments, the single-stranded region comprises random nucleotides. In some embodiments, the random nucleotides in the single-stranded region hybridize to sequences at the 5′ termini of the first population of nucleic acids. In some embodiments, the blocking group comprises at least one uracil base. In some embodiments, the splint region of the splint oligonucleotide comprises at least one uracil base within the first strand of the double-stranded adapter region, within the single-stranded region, or within both. In some embodiments, both strands of the splint oligonucleotide comprise at least one uracil. In some embodiments, both strands of the double-stranded region comprise at least one uracil base and the random region also comprises at least one uracil base. In some embodiments, the selectively attaching a blocking group to the 5′ termini of the first population of nucleic acids is conducted using a ligase to ligate the blocking group to the 5′ termini of the first population of nucleic acids. In some embodiments, a ligase used to ligate the blocking group to the 5′ termini of the first population of nucleic acids is selected from the group consisting of T4 DNA ligase, Splint R ligase, PBCV-1 DNA ligase, ATP-dependent ligase, and Chlorella virus DNA ligase.

In some embodiments, the selectively modifying in (b) comprises treating the first population of nucleic acids with an exonuclease to produce degraded nucleic acids. In some embodiments, the exonuclease is a 5′ phosphorylation-specific exonuclease. In some embodiments, the 5′ phosphorylation-specific exonuclease is Lambda exonuclease or Terminator exonuclease. In some embodiments, the selectively modifying the second population of nucleic acids in (c) comprises modifying 5′ termini of the second population of nucleic acids to become amenable to the second chemical reaction. In some embodiments, the preceding claims, the second chemical reaction is a ligation reaction. In some embodiments, the selectively modifying the second population of nucleic acids in (c) comprises phosphorylating 5′ termini of the second population of nucleic acids. In some embodiments, the phosphorylating the 5′ termini of the second population of nucleic acids is conducted using a kinase. In some embodiments, the kinase is a polynucleotide kinase (PNK). In some embodiments, the method further comprises attaching a 3′-adapter to 3′ termini of nucleic acids within the mixture of nucleic acids. In some embodiments, the 3′-adapter is a splint oligonucleotide. In some embodiments, the method further comprises selectively ligating a 5′-adapter to the nucleic acids of the second population. In some embodiments, the 5′ adapter is a splint oligonucleotide that comprise a double-stranded region and a single-stranded region, wherein a first strand of the double-stranded region is connected to the single-stranded region and a second strand of the double-stranded region is hybridized to the first strand. In some embodiments, the single-stranded region comprises random nucleotides. In some embodiments, the random nucleotides in the single-stranded region hybridize to sequences at the 5′ termini of the second population of nucleic acids. In some embodiments, the splint oligonucleotide comprises at least one uracil base. In some embodiments, a splint region comprises at least one uracil base, wherein the splint region comprises (i) the first strand of the double-stranded region connected to the single-stranded region and (ii) the single-stranded region. In some embodiments, the second strand of the double-stranded region does not comprise an uracil base.

In some embodiments, the method, further comprises selectively ligation a 5′ blocking splint oligonucleotide to the 5′ termini of the first population of nucleic acids, wherein the 5′ blocking splint oligonucleotide comprises (i) a double-stranded region comprising a first strand and a second strand wherein the second strand comprises at least one uracil base and (ii) a single-stranded region connected to the first strand, and the first strand or the single-stranded region comprises at least one uracil base. In some embodiments, the method, further comprises cleaving one or more uracil bases in the blocking group or the splint oligonucleotide. In some embodiments, the one or more uracil bases are cleaved with an uracil DNA glycosylase enzyme to produce one or more abasic sites.

In some embodiments, the method further comprises using an enzyme to break a DNA phosphodiester backbone at the one or more abasic sites. In some embodiments, the selective ligating comprises treating the nucleic acids with a ligase selected from the group consisting of a T4 DNA ligase, Splint R ligase, PBCV-1 DNA ligase, and Chlorella virus DNA ligase. In some embodiments, further comprises performing a PCR reaction that selectively amplifies the second population of nucleic acids. In some embodiments, the PCR reaction is conducted using primers that hybridize to sequences within the 5′ and 3′ splint oligonucleotides.

In some embodiments, the method further comprises performing a Next Generation Sequencing assay that preferentially sequences the second population of nucleic acids. In some embodiments, the sample is a body fluid sample. In some embodiments, the body fluid sample is a sample selected from the group consisting of blood, serum, plasma, bronchial lavage, synovial fluid, bronchoalveolar lavage and cerebrospinal fluid. In some embodiments, the second population of nucleic acids comprises microbial nucleic acids selected from the group consisting of bacterial nucleic acids, fungal nucleic acids, parasite nucleic acids, protozoa nucleic acids and viral nucleic acids. In some embodiments, the sample is from a human subject.

In some embodiments, the method further comprises adding one or more process control molecules to the sample to provide a spiked sample. In some embodiments, the sample is a biological fluid sample; and wherein the nucleic acids are not obtained or extracted from the biological fluid sample before preparing the nucleic acid library. In some embodiments, the method further comprises performing a PCR reaction using primers that hybridize to the 5′ Splint oligonucleotide but not to the blocking group.

In some embodiments, the first population of nucleic acids comprises human nucleic acids and the second population of nucleic acids comprises microbial nucleic acids and the method comprises ligating a first adapter to the 5′ termini of the first population of nucleic acids, and the first adapter is not amenable to PCR amplification; and phosphorylating the 5′ termini of the second population of nucleic acids and then ligating a second adapter to the second population of nucleic acids, and the second adapter is amenable to PCR amplification. In some embodiments, the first population of nucleic acids or the second population of nucleic acids comprise single-stranded nucleic acids. In some embodiments, the first population of nucleic acids or the second population of nucleic acids comprises double-stranded nucleic acids.

In an aspect, the present disclosure provides a method of preparing a nucleic acid library from a sample, the method comprising (a) providing a sample comprising a mixture of a first population of nucleic acids and a second population of nucleic acids, wherein the first population of nucleic acids are amenable to ligation and the second population of nucleic acids are not amenable to ligation, and; wherein the first population of nucleic acids and the second population of nucleic acids are derived from genomes with different genotypes; and (b) selectively enriching for the second population based on its amenability to ligation. In some embodiments, the second population comprises microbial nucleic acids and the first population comprises human nucleic acids. In some embodiments, the first population comprises fetal nucleic acids and the second population comprises maternal nucleic acids, or wherein the second population comprises fetal nucleic acids and the first population comprises maternal nucleic acids. In some embodiments, the first population comprises transplant recipient nucleic acids and the second population comprises transplant donor nucleic acids. In some embodiments, the second population comprises transplant recipient nucleic acids and the first population comprises transplant donor nucleic acids. In some embodiments, the first population comprises mutant nucleic acids and the second population comprises wildtype nucleic acids or wherein the second population comprises mutant nucleic acids and the first population comprises wildtype nucleic acids. In some embodiments, the first population of nucleic acids has 5′ termini that are natively phosphorylated and the second population of nucleic acids has 5′ termini that are natively unphosphorylated.

In one aspect, disclosed herein is a kit comprising: (a) a 5′ splint oligonucleotide comprising (i) a double-stranded adapter region comprising a first adapter strand and a second adapter strand and (ii) a single-stranded region attached to the first strand of the double-stranded adapter region; and (b) a 5′ blocking splint oligonucleotide comprising (i) a double-stranded blocking adapter region comprising a first blocking adapter strand and a second blocking adapter strand and (ii) a single-stranded region attached to the first strand of the double-stranded blocking adapter region, wherein the second blocking adapter strand has a 5′ terminus that is not amenable to ligation or phosphorylation. In some embodiments, the kit further comprises a 3′ splint oligonucleotide comprising (i) a double-stranded adapter region comprising a first adapter strand and a second adapter strand and (ii) a single-stranded random region attached to the first strand of the double-stranded adapter region. In some embodiments, the 5′ terminus of the second blocking adapter strand is not phosphorylated and is resistant to being phosphorylated by a kinase. In some embodiments, the kit further comprises a kinase. In some embodiments, the kit further comprises PNK kinase. In some embodiments, the kit further comprises a ligase. In some embodiments, the 3′ splint oligonucleotide does not comprise a uracil base. In some embodiments, the 5′ splint oligonucleotide does not comprise a uracil base. In some embodiments, the 5′ blocking splint oligonucleotide of the kit comprises at least one uracil base. In some embodiments, the at least one uracil base is present in the single-stranded random region, the first strand of the double-stranded blocking adapter region, or both the single-stranded random region and the first strand of the double-stranded blocking adapter region. In some embodiments, the at least one uracil base of the kit is present in the second strand of the double-stranded blocking adapter region. In some embodiments, the kit further comprises a uracil DNA glycosylase (UDG). In some embodiments, the kit further comprises an endonuclease. In some embodiments, the endonuclease is DNA glycosylase-lyase Endonuclease VIII. In some embodiments, the single-stranded region of the 5′ splint oligonucleotide of the kit, the 5′ splint blocking oligonucleotide, the 3′ splint oligonucleotide, or any combination thereof, comprises a random N-mer region.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A and FIG. 1B illustrate a process of generating a single-stranded nucleic acid library from mixtures of nucleic acids (cell-free nucleic acids, e.g. cell-free DNA) in a sample (FIG. 1A). The dotted boxes around reactions in FIG. 1A are detailed further in FIG. 1B.

FIG. 2A and FIG. 2B illustrate a human fragment length distribution after single-stranded library preparation in a ligation-based process (FIG. 2A) and in a template-shift process (FIG. 2B). Both single-stranded library preparation methods show a 50 bp peak, in addition to the nucleosome at greater than 60 bp, though both vary in clinical samples.

FIG. 3 shows the differential phosphorylation of human cell-free DNA (and spiked pathogen DNA) versus native, microbial cell-free DNA. Plasma from healthy human subjects was spiked with pathogen DNA. Spiked pathogens include Aspergillus fumigatus, Cryptosporidium parvum, and Staphylococcus aureus. Endogenous microbes are shown by the thick horizontal bar and include Streptococcus thermophilus, Heliobacter pylori, and Haemophilus influenza. Duplicate reads, assumed to be derived from PCR duplication or sequencing instrument error, are identified based on alignment, and removed in a process referred to as deduping. As a result of this process, the count of estimated unique or deduped reads is obtained by mapping to a particular pathogen reference. The relative abundance of microbes is expressed as estimated deduped reads (EDR), or reads per million (RPM, normalized to total reads for the sample), or reads per volume of sample (MPM, microbes per microliter). The ratio of microbial cfDNA (estimated deduped reads, EDR) to total human reads (EDR/ddHuman) is given without (no PNK, right) and with phosphorylation by PNK (control, left). The results show a 3-5× depletion of microbial cell-free DNA in the absence of PNK phosphorylation.

FIG. 4 shows results after a nucleic acid library is prepared using dummy oligonucleotides to preferentially enrich for microbial cell-free DNA extracted from healthy human plasma. In each graph, the four left-most data values reflect EDR/ddHuman after using dummy oligonucleotides during the sample processing. Spiked pathogens including Staphylococcus aureus, Mycobacterium tuberculosis, and Aspergillus fumigatus (upper panels), were detected with a large reduction in sensitivity when dummy oligo's were used. In contrast, endogenous pathogens such as Heliobacter pylori, Streptococcus thermophilus, and Haemophilus influenzae (lower panel), were detected with a three-fold increase in sensitivity by EDR/ddHuman.

FIG. 5A, FIG. 5B, FIG. 5C, FIG. 5D, FIG. 5E, and FIG. 5F show the enrichment obtained from testing mcfNA in plasma samples of patients who are known to be infected by a microorganism using the method disclosed herein with dummy oligos. FIG. 5A shows results with Staphylococcus aureus in plasma of S. aureus-infected patients. S. aureus shows 2.7× EDR/ddHuman enrichment with a dummy oligo v. without a dummy oligo. Gray dots are SA, black dots are other microbial organisms in the samples. FIG. 5B are results with Aspergillus in plasma of Aspergillus-infected patients. Improvements were not initially found in plasma from patients with aspergillus infections. Aspergillus shows no EDR/ddHuman enrichment (or depletion) for a dummy oligo v. without a dummy oligo. Gray dots are Aspergillus, black dots are other microorganisms in the samples. FIG. 5C are results with BRIC in BRIC-patients. BRIC is a set of pathogens often seen in immune compromised patients. BRIC pathogens show 2×EDR/ddHuman enrichment with a dummy oligo v. without a dummy oligo. FIG. 5D shows improvement in sensitivity seen generally in plasma from viral-infected patients (3.1-fold improvement). FIG. 5E shows improvement in sensitivity was seen generally in plasma from bacteria-infected patients (2.6-fold improvement). FIG. 5F shows improvement in sensitivity was seen generally in plasma from eukaryote-infected patients (1.4-fold improvement).

FIG. 6 shows a computer control system that is programmed or otherwise configured to implement methods provided herein.

### DETAILED DESCRIPTION

The following passages describe different aspects of the disclosure in greater detail. Each aspect, embodiment, or feature of the disclosure may be combined with any other aspect, embodiment, or feature the disclosure unless clearly indicated to the contrary.

### Overview

Disclosed herein, in some embodiments, are methods and kits to distinguish or enrich populations of nucleic acids in a sample (e.g., bodily fluid sample, plasma sample, serum sample, broncho-alveolar lavage sample, blood sample, etc.). Generally, the methods and kits are for use in preparing a nucleic acid sequencing library that is enriched for a particular population of nucleic acids. In some cases, the methods disclosed can be used to enrich for populations of nucleic acids based on the source of the nucleic acids (e.g., human host vs. microbe; transplant donor vs. recipient; cancerous vs. non-cancerous; fetal vs. maternal, etc.). In some embodiments, the methods disclosed herein may be used to distinguish populations of nucleic acids comprising genomes with different genotypes, such as a human host versus a microbe.

In some embodiments, the methods may be used to distinguish between two populations of nucleic acids based on the amenability or susceptibility of the nucleic acids to a particular chemical reaction. For example, as shown herein, microbial cell-free nucleic acids (e.g., microbial cell-free DNA (or “mcfDNA”)) tend to lack phosphorylated 5′ termini, in contrast with human cell-free nucleic acids (e.g., cell-free DNA), which tend to contain phosphorylated 5′ termini. (In some cases, synthetically-produced DNA, such as synthetically produced spike-in DNA also contains phosphorylated 5′ termini.) As such, human cell-free nucleic acids are naturally more amenable to certain chemical reactions such as ligation reactions, than microbial cell-free nucleic acids. This is because, generally, ligation reactions can involve ligating the phosphorylated 5′ terminus of one nucleic acid to the 3′ (unphosphorylated) terminus of a second nucleic acid. The 5′ termini of microbial cell-free nucleic acids thus tend to be resistant to ligation reactions unless they are further altered, such as by phosphorylating the 5′ termini with a kinase such as polynucleotide kinase (PNK).

In some cases, this disclosure provides sample processing methods that rely on a series of reversals. The method may involve providing a sample comprising a mixture of two different populations of nucleic acids, which can be differentiated in that one of the populations contains a chemical feature lacking in the other. In some instances, the chemical feature is a feature that permits the population of nucleic acids to be susceptible to a particular chemical reaction, such as a ligation reaction, a PCR reaction, a kinase reaction, or a sequencing reaction. The method may then involve selectively targeting the population with the chemical feature in order to block, inactivate or remove the chemical feature. The method may then involve adding or attaching the chemical feature to the population lacking the feature, so that the two populations have effectively switched places. The mixture of nucleic acids may then be subjected to a chemical reaction, such that the population newly modified to contain the chemical feature is preferentially enriched (e.g., by being preferentially ligated to an adapter, preferentially amplified, and/or preferentially sequenced).

In some embodiments, the methods provided herein involve obtaining a mixture of two populations of nucleic acids and selectively modifying a first population of nucleic acids (e.g., human nucleic acids) by reducing or eliminating its amenability to a chemical reaction (e.g., a ligation reaction) followed by selectively modifying a second population of nucleic acids (e.g., microbial cell-free nucleic acids) in order to enhance its amenability to a chemical reaction (e.g., a ligation reaction). The methods allow selective enrichment of a population of nucleic acids (e.g., mcfDNA) by selectively targeting nucleic acids based on differential phosphorylation, or other chemical difference.

In some instances, a first population of nucleic acids (e.g., human cell-free nucleic acids, synthetic spike-in nucleic acids) are altered by attaching them to a blocking group or oligonucleotide that selectively attaches to 5′ phosphorylated termini, e.g., by a ligation reaction.

In some cases, the blocking group (also referred to herein as a “dummy oligo,” or a “decoy oligo”) may comprise a feature that renders the first population of nucleic acids impervious to a reaction, such as a ligation reaction or an amplification reaction. For example, a 5′ blocking group can be designed to lack a 5′ phosphate group and is thus impervious to additional ligation reactions after being ligated to the first population of nucleic acids. The 5′ blocking group can also be designed to have a 5′ terminus that is not capable of being phosphorylated by a kinase. Ligation of such a 5′ blocking group to a first population of nucleic acids (e.g., human cell-free DNA) prevents the 5′ termini from engaging in further ligation reactions, such as being ligated to an adapter (e.g., a sequencing adapter). In such cases, if the sequencing adapter is being used to amplify the nucleic acids (e.g., by using a primer that is specific for the sequencing adapter), any nucleic acids that are attached to the blocking group will not participate in the ligation reaction, will not attach to a 5′ sequencing adapter, and thus will be prevented from being amplified or being used in downstream processes such as a sequencing assay.

After introduction of the blocking group, the mixture of nucleic acids may be subjected to a reaction that causes the second population of nucleic acids (e.g., mcfDNA) to become amenable to a chemical reaction. For example, the 5′ termini of the nucleic acids can be phosphorylated with a kinase such as polynucleotide kinase (PNK), rendering the mcfDNA amenable to a ligation reaction. Then, when 5′ adapters are added to the mixture of two populations of nucleic acids, the adapters selectively attach to the second population (e.g., the mcfDNA) over the first population (e.g., human cell-free DNA) given that the blocking group attached to the first population remains unphosphorylated even after addition of the kinase. Following amplification the second population of nucleic acids is selectively enriched over the first population.

In some cases, the blocking group is used to tag or identify a population of nucleic acids in a mixture of populations. For example, a blocking group containing a known sequence can be used to tag and identify 5′ phosphorylated nucleic acids (e.g., human cell-free DNA) within the mixture. In such cases, the blocking group may or may not be amenable to a further ligation reaction to a 5′ adapter. In some cases, where a blocking group that is not amenable to further ligation is used, the known sequence of the blocking group can be used to determine whether any first population nucleic acids (e.g., human cell-free DNA) ended up being sequenced in a later sequencing assay. In such case, the tag can be used as an internal control to assess the quality and efficiency of the blocking mechanism. In other cases, where a blocking group that is amenable to further ligation is used, then the known sequence tagged to the first population nucleic acids (e.g., human cell-free DNA) can be later used to identify human cell-free DNA in downstream processes such as NGS.

In some cases, the first population may be selectively degraded based on its chemical difference from the second population. For example, in cases where the first population is more highly phosphorylated at its 5′ termini (e.g., human cell-free DNA) than the second population, the first population can be selectively degraded using a 5′ phosphorylation-specific exonuclease (e.g., Lambda exonuclease or Terminator exonuclease). Selective degradation of the first population may result in selective enrichment of the second population of cfDNA, which can go on to be PCR amplified or be subjected to other downstream processes (e.g., next-generation sequencing, massively-parallel sequencing).

In some cases, the methods provided herein are particularly useful to analyze single-stranded nucleic acids. In some cases, the methods may involve denaturing a mixture of nucleic acids to obtain single-stranded nucleic acids (e.g., single-stranded DNA). Adapters such as splint oligonucleotides may be used to enrich for a population of nucleic acids in the mixture of single-stranded nucleic acids. Generally, as provided herein, a splint oligonucleotide comprises a double-stranded region and a single-stranded region. The single-stranded region may contain random nucleotides or N-mers that randomly hybridize to genomic sequences within the mixture. The double-stranded region can be considered an adapter region containing a first strand and a second strand. The first strand may be connected to the single-stranded region, while the second strand may be hybridized to the first strand. The “splint” region of the oligonucleotide contains the first strand connected to the single-stranded region.

The methods provided herein may involve use of a 3′ splint oligo, a 5′ splint oligo and/or a 5′ splint blocking group (or 5′ dummy oligo). In some cases, the 5′ splint blocking group comprises a single-stranded region preferably comprising random nucleotides that have agnostic binding activity. The 5′ splint blocking group may comprise a splint region that comprises at least one uracil base in the random region, in the first strand, or in both the random region and the first strand. In some cases, the second strand of the double-stranded region comprises at least one uracil base. In some embodiments, the 5′ dummy (decoy) oligo is ligated to natively phosphorylated 5′ termini of the first population, but not to the second population which is not amenable to ligation. In some cases, the 3′ splint oligo and/or the 5′ splint oligo also comprise one or more uracil bases. In some cases, the uracil bases are present in the splint region (e.g., the single stranded region or the first strand of the double-stranded region). In some cases, in these splint oligo's, uracil bases are not present in the second strand of the double-stranded region.

In some cases, the method comprises subjecting the sample to a uracil deglycosylase (UDG) enzyme or USER digestion in order to cleave the uracil bases within the splint oligo's or splint blocking groups. In some cases, the USER digestion removes the splint regions of the splint oligo's and splint blocking groups. In some cases, the USER digestion also removes the entire splint blocking group, including the strand directly ligated to the 5′ end of the first population (e.g., human cell-free DNA).

This disclosure provides, in some embodiments, kits, particularly kits that can be used to distinguish or enrich for different populations of nucleic acids within a mixture of nucleic acids. In some cases, the kits comprise: (a) a 5′ splint oligonucleotide comprising (i) a double-stranded adapter region comprising a first adapter strand and a second adapter strand and (ii) a single-stranded region attached to the first strand of the double-stranded adapter region; and (b) a 5′ blocking splint oligonucleotide comprising (i) a double-stranded blocking adapter region comprising a first blocking adapter strand and a second blocking adapter strand and (ii) a single-stranded region attached to the first strand of the double-stranded blocking adapter region, wherein the second blocking adapter strand has a 5′ terminus that is not amenable to ligation. In some cases, the kit further comprises a 3′ splint oligonucleotide comprising (i) a double-stranded adapter region comprising a first adapter strand and a second adapter strand and (ii) a single-stranded random region attached to the first strand of the double-stranded adapter region.

### Dummy Oligonucleotides Process

This disclosure provides methods of sample processing to produce nucleic acid sequencing libraries that are preferentially enriched for a certain population of nucleic acids. In general, the methods may involve processing of a sample that comprises mixtures of nucleic acids (e.g., mixtures of cell-free nucleic acids), and the methods herein may be used to distinguish or enrich for different populations within the mixture.

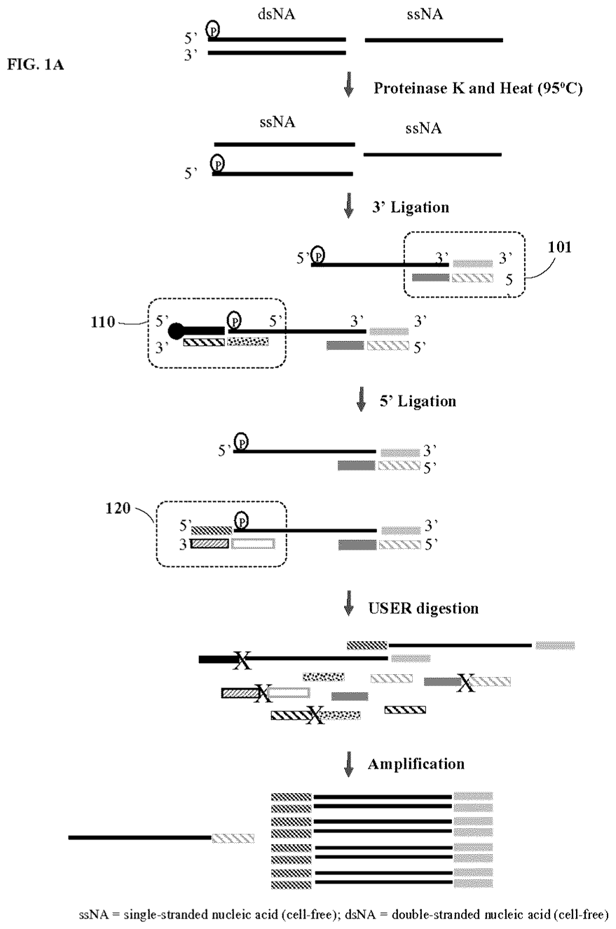

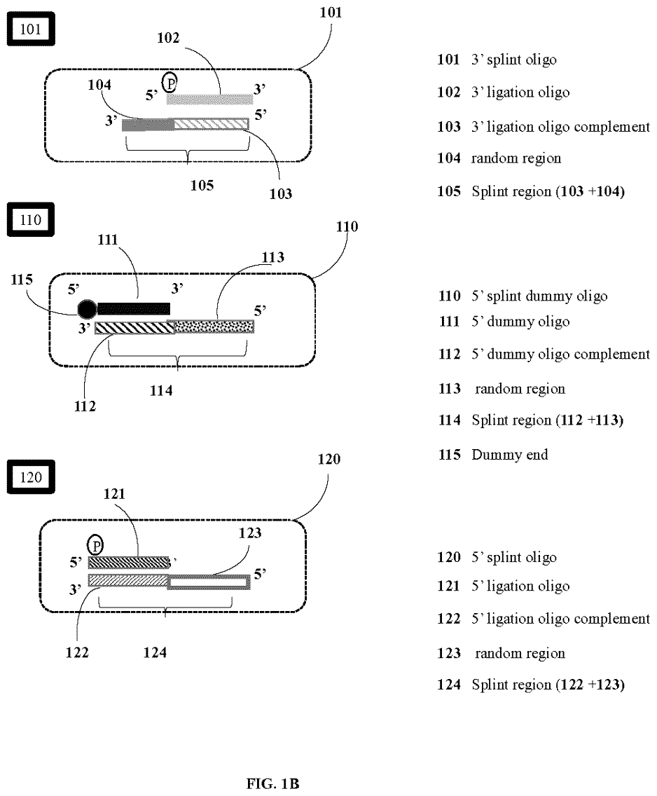

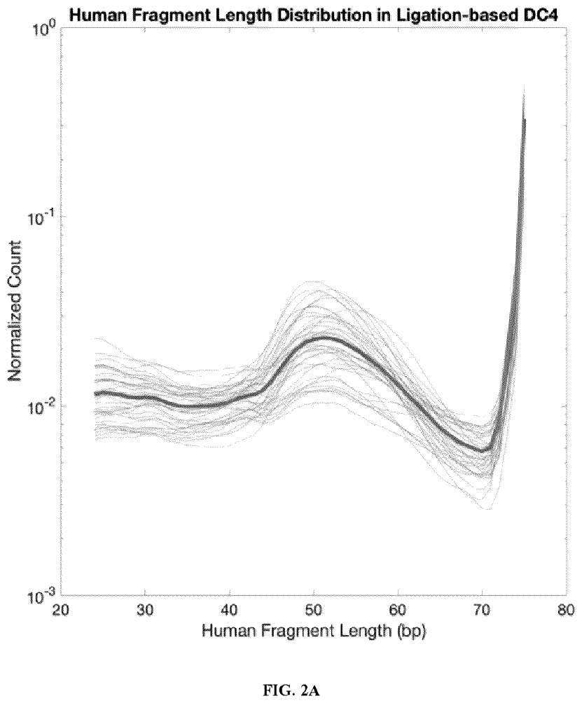

FIG. 1A provides an example of a process for preparing a nucleic acid library enriched for a particular population of nucleic acids. In this example, the sample comprises a mixture of nucleic acids in which some of the nucleic acids have 5′ termini that are phosphorylated, while some of the nucleic acids have 5′ termini that are not phosphorylated. The methods are particularly useful to enrich for target nucleic acids such as microbial cell-free nucleic acids that are less likely to be natively phosphorylated at their 5′ termini compared to the other nucleic acids in the mixture (e.g., human cell-free DNA). However, the basic concept in FIG. 1A can also be applied to chemical differences other than phosphorylation (e.g., methylation, etc.), as well as to mixtures comprising transplant donor and recipient DNA/RNA, fetal and maternal DNA/RNA, mutant and wild-type DNA, etc. In some embodiments, the initial sample is a mixture comprising double-stranded, single-stranded, or a mixture of double-stranded and single-stranded nucleic acids. In some embodiments, a portion of the nucleic acids are phosphorylated at the 5′ end, and a portion are not.

In some embodiments, to remove proteins and to denature nucleic acids (e.g., DNA) into single strands, the sample may be subjected to proteinase K digestion and/or a high temperature. In some cases, the sample may be spiked with a known concentration of control DNA (also referred to herein as “process control molecules”). In some cases, the sample may not be spiked with any control DNA.

In some embodiments, in order to attach an adapter oligonucleotide to the 3′ end of the nucleic acids (e.g., cell-free DNA), a 3′ splint oligonucleotide (“3′ splint oligo” or 3′ adapter) 101 is added to the sample; and a ligation reaction is performed in order to ligate the 3′ splint oligo 101 to the 3′ end of the single-stranded DNA in the sample. The ligation reaction may be performed using a DNA or RNA ligase. Examples of ligases that can be used include, but are not limited to, T4 DNA ligase, a SplintR ligase, a PBCV-1 DNA ligase or a Chlorella virus DNA ligase.

As shown in FIG. 1B, the 3′ splint oligo 101 can contain (a) a double-stranded region (102, 103) that comprises a 3′ ligation oligo 102 and a 3′ ligation oligo complement 103 and (b) a single-stranded region 104 (or overhang region) containing a random sequence of nucleotides (“random region”), wherein the single-stranded region 104 is connected to the 3′ ligation oligo complement 103. Stated a different way, the 3′ splint oligo 101 can contain a “splint” region 105 that contains a random region 104, that is connected, at its 5′ end, to the 3′ ligation oligo complement 103. In some embodiments, the random regions in a collection of 3′ splint oligo's can enable the splint oligo's to bind to a large number of different target nucleic acids in a sample (e.g., target genomic cell-free nucleic acids). FIG. 1B depicts a 3′ ligation oligo 102 that is phosphorylated at its 5′ end to permit ligation to the 3′ end of the cell-free DNA. In some embodiments, the splint region, 105, generally can comprise one or more uracil bases that are susceptible to digestion by USER enzyme. As further described herein, in some cases, the splint region 114 of the 5′ splint dummy oligo and the splint region 124 of the 5′ splint oligo, can also have one more uracil bases that are susceptible to digestion by USER enzyme.

As shown in FIG. 1A, a 5′ ligation reaction can be performed to selectively modify the 5′ phosphorylated ends of the human cell-free DNA with a blocking oligonucleotide (e.g., “dummy oligo”). In some cases, an oligo with an identifiable sequence is used instead of a dummy oligo, along with a dummy oligo or as part of the dummy oligo. In some cases, the oligo with the identifiable sequence can be used to mark or identify a particular sequence, for example, to mark a sequence as “human.” The 5′ ligation can occur before, concurrently, or following the 3′ ligation of the 3′ splint oligo. As shown in FIG. 1B, the 5′ dummy oligo 110 generally has the same structure as the 3′ splint oligo 101 in that it has a double-stranded region (111-112) and a single-stranded region (or overhang region) containing a random sequence of nucleotides 113. In the splint region of the 5′ dummy oligo 114 the random sequence 113 is connected at its 3′ end to the 5′ dummy oligo complement 112. In order to prevent subsequent ligation, the 5′ dummy oligo (or 5′ dummy ligation oligo) is not phosphorylated at its 5′ end (depicted by a solid circle 115) and is also resistant to phosphorylation, e.g., by a polynucleotide kinase (PNK). In some cases, the dummy oligo is resistant to phosphorylate because it contains a blocking group at its 5′ end. In some cases, it is chemically modified to resist the action of a kinase. In some cases, the dummy oligo is attached to a protein (e.g., biotin or avidin) or bead that enables physical removal of the 5′ phosphorylated nucleic acids.

In some cases, as shown in FIG. 1A, a kinase (e.g., polynucleotide kinase (PNK), T4 PNK) is introduced to the sample in order to phosphorylate the 5′ termini of the cell-free nucleic acids, except for the termini of the cell-free DNA blocked by the 5′ dummy oligo. As a result, microbial cell-free DNA is preferentially phosphorylated at the 5′ end, while the dummy oligo selectively prevents the human cell-free DNA from becoming phosphorylated at its 5′ end. In some embodiments, a 5′ splint oligo 120 is then added to the sample. The 5′ splint oligo 120 generally has the same structure as the 5′ dummy splint oligo 110 except that its 5′ end does not need to be resistant to phosphorylation. As shown in FIG. 1B, the 5′ splint oligo 120 has a double stranded region (121-122) and a single-stranded region (or overhang region) containing a random sequence of nucleotides 123. The splint region (124) contains a random sequence of nucleotides 123 (or random region) connected at its 3′ end to the 5′ ligation oligo complement 122.

In some embodiments, the 5′ splint oligo is then subjected to a ligation reaction that preferentially ligates the 5′ splint oligo to cell-free DNA that is not attached to the dummy oligo (in other words, cfDNA that is preferentially microbial cfDNA and that was subjected to phosphorylation by PNK).

In some cases, the nucleic acids are subjected to a USER digestion reaction to cleave the uracil bases (indicated by an “X” in FIG. 1B) in the splint oligo's in order to yield single-stranded DNA that is attached to 5′ and 3′ single-stranded ligation oligos (or adapters). In some cases, the USER digestion cleaves the 5′ dummy oligo (111) containing uracil bases. Next, in some instances, the nucleic acids in the sample are PCR amplified using primers that recognize the 5′ ligation oligo (121) and the 3′ ligation oligos (102) (or 5′ and 3′ adapters). In some cases, additional adapters are attached to the 5′ ligation oligo and/or the 3′ ligation oligo and the PCR amplification is conducted using primers that recognize the additional adapters. The result is a library preferentially enriched for mcfDNA and fully amenable to downstream processing including sequencing and sequence analysis as disclosed herein. In some cases, in which an identifying sequence is added as part of (or instead of) the dummy oligo, human (or natively 5′ phosphorylated) nucleic acids may be identified during the sequencing analysis.

### Initial Samples and Raw Biological Samples

The disclosed methods, systems, compositions, and kits can be used for the analysis of a wide range of different sample types. The disclosure may be particularly useful in the evaluation of initial samples in which the nucleic acids are of low quality or quantity by allowing analysis of a larger fraction of the nucleic acids present in the initial sample, regardless of purification efficiencies or biases or chemical type or structure.

In some embodiments, the initial sample may comprise a raw biological sample. In some embodiments, the initial sample may comprises a solid or a body fluid such as blood, plasma, serum, cerebrospinal fluid, synovial fluid, bronchoalveolar lavage, urine, stool, saliva, abdominal fluid, ascites fluid, peritoneal lavage, gastric fluid, interstitial fluid, lymph fluid, bile, abscess fluid, tissue, amniotic fluid, meconium, sinus aspirate, lymph node, bone marrow, hair, nails, cheek swab, skin swab, urethral swab, cervical swab, nasopharyngeal swab, nasopharyngeal aspirate, vaginal swab, epithelial cells, semen, vaginal discharge, intercellular fluid, pericardial fluid, rectal swab, bone, skin tissue, soft tissue, tears, and/or a nasal sample. In some embodiments, the initial sample comprises a solid or a body fluid selected from the group consisting of plasma, cerebrospinal fluid, bronchoalveolar lavage, urine, and synovial fluid. In some embodiments, the initial sample comprises plasma. In some embodiments, the initial sample comprises, consists of, or consists essentially of urine. In some embodiments, the initial sample comprises cerebrospinal fluid. In some embodiments, the initial sample is from a human subject. In some embodiments, an initial sample may comprise circulating tumor or fetal nucleic acids. In some embodiments, the initial sample comprises circulating donor nucleic acids.

Cell-free nucleic acids may be present in any biological sample, including raw biological samples, raw samples, and initial samples. In some embodiments, an initial sample can be made up of, in whole or in part, cells and/or tissue. The initial sample may be cell-free or cell-depleted. The initial cell-free sample or initial sample may comprise nucleic acids that originated from a different site in the body, such as a site of pathogenic infection. In the case of blood, serum, lymph, or plasma, the initial sample may contain “circulating” cell-free nucleic acids that originated at anatomic locations other than the site of bodily fluid collection of the fluid in question. The cell-free samples or cell-depleted initial samples can be obtained by depleting or removing cells, cell fragments, or exosomes by a known technique such as by centrifugation or filtration.

In some embodiments, substances that may affect library generation are partially or completely removed. In some embodiments, the nucleic acid library is generated from the initial sample without prior partial or complete removal of any substance that may affect the library yield or inhibit the library generation. Examples of substances that may affect library generation that can be completely or partially removed include, but are not limited to, heparin or other oligo-/poly-saccharides, EDTA, fat, lipids, fatty acids, urea, haemoglobin, and other products of hemolysis, immunoglobulin, lactoferrin, buffy coat, components of the buffy coat, calcium, collagen, haemitin, tannic acid, melanin, humic acids, antiviral substances (e.g., acyclovir), therapeutic drugs, human serum albumin, lipoproteins, triglyceride-rich lipoproteins, hemolysate, protein, conjugated bilirubin, unconjugated bilirubin, antibody, acetylcysteine, ampicillin, cefoxitin, doxycycline, theophylline, levodopa, methyldopa, metronidazole, acetylsalicylic acid, ibuprofen, phenylbutazone, rifampicin, cyclosporine, acetaminophen, creatinine, glucose, glycerol, lactate, pyruvate, uric acid, and/or biotin.

### Subjects

An initial sample can be derived from any subject (e.g., a mammal such as a human subject, a non-human subject, etc). The initial sample may comprise a bodily fluid e.g., a plasma, or other bodily fluid. The subject can be healthy. In some embodiments, the subject is a human host. In some embodiments, the subject is a human patient having, suspected of having, or at risk of having, a disease or infection.

The initial sample can be from a subject who has a specific disease, condition, or infection, or is suspected of having (or at risk of having) a specific disease, condition, or infection. For example, the initial sample can be from a cancer patient, a patient suspected of having cancer or a patient at risk of having cancer. In some embodiments, the initial sample can be from a patient with an infection, a patient suspected of an infection, or a patient at risk of having an infection. In some embodiments, the initial sample is from a subject who has undergone, or will undergo, an organ transplant. In some embodiments, the initial sample is obtained from a transplant recipient.

A human subject can be a male or female. In some embodiments, the sample can be from a human embryo or a human fetus. In some embodiments, the human can be an infant, toddler, child, teenager, adult, or elderly person. In some embodiments, the subject is a female subject who is pregnant, suspected of being pregnant, or planning to become pregnant. In some embodiments, the female subject is not pregnant, or is not suspected of being pregnant or is not planning to become pregnant.

In some embodiments, the subject is a human subject who has undergone an organ transplant or who is planning to undergo organ transplant.

In some embodiments, the subject is a farm animal, a lab animal, a domestic pet, or any other animal. For example only, in some embodiments, the animal can be a primate, a rodent, an insect, a dog, a cat, a horse, a cow, a mouse, a rat, a pig, a fish, a bird, a chicken, or a monkey.

In some embodiments, the subject has a genetic disease or disorder, is affected by a genetic disease or disorder, or is at risk of having a genetic disease or disorder. A genetic disease or disorder can be linked to a genetic variation such as mutations, insertions, additions, deletions, translocations, point mutations, trinucleotide repeat disorders, single nucleotide polymorphisms (SNPs), or a combination of genetic variations. In some cases, the subject has or is suspected of having a cancer.

### Nucleic Acids

The disclosure provides for the detection, and genetic analysis of various chemical and structural forms of nucleic acid found in a biological sample. Detection of various chemical and structural forms of nucleic acid found in a biological sample may be concurrent, consecutive, or independent. Nucleic acids can include various chemical forms of a DNA molecule as well as various chemical forms of an RNA molecule. Nucleic acids can also include different structural forms of DNA and RNA found in a sample. In some embodiments, the nucleic acids can be located outside of cells. In some cases, the nucleic acids are derived from viral particles or spores. In some cases, the nucleic acids are cell-free nucleic acids. In some cases, the nucleic acids are circulating cell-free nucleic acids.

Nucleic acids may be any type of nucleic acid including but not limited to: double-stranded (ds) nucleic acids, single stranded (ss) nucleic acids, DNA, RNA, cDNA, mRNA, cRNA, tRNA, ribosomal RNA, dsDNA, ssDNA, miRNA, siRNA, circulating nucleic acids, circulating cell-free nucleic acids, circulating DNA, circulating RNA, cell-free nucleic acids, cell-free DNA, cell-free RNA, circulating cell-free DNA, cell-free dsDNA, cell-free ssDNA, circulating cell-free RNA, genomic DNA, exosomes, cell-free pathogen nucleic acids, circulating microbe or pathogen nucleic acids, mitochondrial nucleic acids, non-mitochondrial nucleic acids, nuclear DNA, nuclear RNA, chromosomal DNA, circulating tumor DNA, circulating tumor RNA, circular nucleic acids, circular DNA, circular RNA, circular single-stranded DNA, circular double-stranded DNA, linear nucleic acids, linear DNA, linear RNA, linear single-stranded DNA, linear double-stranded DNA, plasmids, bacterial nucleic acids, fungal nucleic acids, parasite nucleic acids, viral nucleic acids, cell-free bacterial nucleic acids, cell-free fungal nucleic acids, cell-free parasite nucleic acids, viral particle-associated nucleic acids, mitochondrial DNA, intercellular signal nucleic acids, exogenous nucleic acids, DNA enzymes, RNA enzymes, food-derived nucleic acids, any metabolic form of nucleic acid-based therapeutics, or any combination thereof.

Nucleic acids may be nucleic acids derived from microbes or pathogens including but not limited to viruses, bacteria, fungi, parasites, and any other microbe, particularly an infectious microbe or potentially infectious microbe. Nucleic acids may derive from archaea, bacteria, fungi, molds, prokaryotes, protists, protozoa, eukaryotes, and/or viruses. In some embodiments, nucleic acids may be derived directly from the subject, as opposed to a microbe or pathogen.

In some embodiments, the present disclosure provides for generation of a single-stranded nucleic acid library. The single-stranded methods provided by the present disclosure can be applied for more efficient processing of shorter nucleic acid fragments as well as less biased processing of nucleic acids in respect to any of their properties (e.g., nucleic acid length, sequence, GC content, secondary and/or any higher order structure, degree of damage, such as nicking and/or the presence of gaps, and/or degree of chemical damage). In some embodiments, the single-stranded nucleic acid methods, composition, systems, and kits can be applied for a microbe or pathogen identification in samples that contain circulating or cell-free nucleic acids or highly degraded or low-quality samples such as ancient, formalin-fixed paraffin-embedded (FFPE) samples, or samples which have undergone many freeze-thaw cycles. In some embodiments, the present disclosure provides for analysis of both double-stranded and single-stranded nucleic acids in a sample. In some embodiments, double-stranded nucleic acids are denatured to form single-stranded nucleic acids.

In some embodiments, the sample can comprise cell-free DNA and RNA (e.g., RNA or capsid-protected RNA). In such cases, the method can comprise obtaining denatured cell-free DNA as described herein, while also capturing RNA sequences. Since RNA is less stable than DNA, it may be desirable to convert the RNA to DNA using a reverse transcriptase, as further described herein. As a result of this process, the sample may be enriched for microbial cell-free DNA using the dummy oligomer processes described herein, while still comprising cDNA derived from the RNA in the sample.

Previous efforts had indicated heat denaturation of samples reduced RNA fragment recovery in the library generation process. Heat in the presence of divalent cations (Ca²⁺ or Mg²⁺) causes strand cleavage of RNA molecules. Previous efforts to solve this problem by chelating the divalent cations have been attempted, but chelating the divalent cations is difficult to achieve given the reaction buffer requirements or variable concentration of anticoagulants in the input samples. In some embodiments, a sample comprising cell free or viral particle protected RNA is incubated with reverse transcriptase. The cell free RNA is converted to cDNA which is stable in subsequent heat denaturation steps. The heat denaturation step releases particle protected RNA A second incubation with reverse transcriptase converts the released nucleic acids to cDNA. In some aspects of the method, a polyadenylation process occurs before incubation with the reverse transcriptase. In some aspects of the method a thermolabile or deactivatable proteinase K is used. In some aspects, salts are removed or reduced to destabilize dsDNA.

In addition to reducing environmental contamination protease incubation may reduce inhibitors of the direct to library process. Incubating with a protease may include any protease known in art including but not limited to proteinase K. In the presence of detergents and shearing forces proteases such as proteinase K may release nucleic acids present in viral capsids, bacterial and eukaryotic cells. The released nucleic acids may be accessible for utilization in downstream library preparation. If the target nucleic acid is cell free RNA, the method will need a variation to convert cfRNA to cDNA prior to incubation with protease, particularly proteinase K, and heat denaturation. The protease and denaturation step should be followed by additional steps to capture the newly released RNA along with cfDNA and cDNA present in the sample. The additional step may be A-tailing of the nucleic acid or a second round of cDNA synthesis. The additional step enables capture of particle protected and cell-free RNA in the same protocol.

In some embodiments, the subject may have, or is suspected of having, a pathogenic infection. In some embodiments, the sample from the host subject comprises host DNA and RNA, as well as DNA and RNA from a pathogen or microbe which can be in the chemical or structural form of ssRNA, ssDNA, dsRNA, or dsDNA.

### Denaturation

The disclosure may include the step of denaturing nucleic acids. Denaturation may cause all, most, part, or a sufficient part for detection, of the double-stranded nucleic acids to become single-stranded. Denaturation may occur at any step in the process. In some embodiments, denaturation may remove all, most, or part of the secondary, tertiary, or quaternary structure of double-stranded or single-stranded nucleic acids. As such, any type of initial sample may be subjected to the denaturation step, including samples that contain, or are suspected to contain, only double-stranded nucleic acids, only single-stranded nucleic acids, a mixture of double-stranded and single-stranded nucleic acids, or any higher order nucleic acid structure.

The nucleic acids may be denatured using any method known in the art. In some embodiments, the nucleic acids are denatured using heat. In some embodiments, single-stranded nucleic acids in the sample arise as a result of being subjected to denaturation. In some embodiments, however, the nucleic acids in the sample are single-stranded because they were originally single-stranded when they were obtained from the subject, e.g., without limitation, as single-stranded viral genomic RNA, or single-stranded DNA or as a result of shipping and handling conditions.

In some embodiments, denaturation is accomplished by applying heat to the sample for an amount of time sufficient to denature double-stranded nucleic acids of interest or to denature secondary, tertiary, or quaternary structures of double-stranded or single-stranded nucleic acids. In general, the sample may be denatured by heating at 95° C., or within a range from about 65 to about 110° C., such as from about 85 to about 100° C. Similarly, the sample may be heated at any temperature between about 50° C. and about 110° C. for any length of time sufficient to effectuate the denaturation, e.g., from about 1 second to about 60 minutes. In some embodiments, long nucleic acids such as intact dsRNA viruses may require longer denaturation times. In general, denaturation is performed in order to ensure that all, most, or part of the nucleic acids or nucleic acids of interest within a sample are present in single-stranded form.

In some embodiments, denaturation comprises denaturation to enrich certain nucleic acids. In some embodiments, selective denaturation comprises one or more denaturation steps effective for the selection of fragments of a certain length and/or GC-content. In some embodiments, selective denaturation comprises incubation at selected or elevated temperatures. In some embodiments, the selective denaturation step comprises incubation at a temperature of about 45° C., at a temperature of about 50° C., at a temperature of about 55° C., at a temperature of about 60° C., at a temperature of about 65° C., at a temperature of about 70° C., at a temperature of about 75° C., at a temperature of about 80° C., at a temperature of about 85° C., at a temperature of about 90° C., at a temperature of about 95° C., at a temperature of about 100° C., at a temperature of about 105° C., at a temperature of about 110° C. In some embodiments, setting the temperature occurs at any of the denaturation steps such as, for example, without limitation, following dephosphorylation, preceding 3′-end adapter attachment, and/or during an elution step.

In some embodiments, denaturation may remove all, most, part, or a sufficient part for detection of the secondary, tertiary, or quaternary structures in single-stranded DNA and/or RNA molecules. Non-limiting examples of domains of secondary structure that may be removed during the denaturation step include hairpin loops, hairpin stems, bulges, internal loops, and complexes of complementary nucleic acid sequences and any element contributing to folding of the molecule or complexes. In some embodiments, denaturation may not need to be performed, for example when the sample is known to contain only single-stranded nucleic acids or when there is a desire to restrict the ultimate analysis to only the single-stranded and not the double-stranded nucleic acids in the sample.

### Chemical and Mechanical Denaturation

In some embodiments, denaturation comprises adding one or more denaturing agents for a selective or controlled denaturation. In some embodiments, denaturation comprises a selective or controlled denaturation. Depending on the application, chemical or mechanical denaturation can be used (e.g., sonication, mechanical force applied by magnetic field (e.g., magnetic tweezers) or optical traps (e.g., optical tweezers) or the like) with the methods.

Chemical denaturation agents that can be used with the methods of the disclosure include but are not limited to, alkaline agents (e.g., NaOH), formamide, guanidinium chloride, guanidine, sodium salicylate, dimethyl sulfoxide (DMSO), propylene glycol, betaine, or urea. In some embodiments, the one or more denaturing agents comprises for example, without limitation, one or more of formamide, urea, guanidinium chloride, salts, betaine, detergents, surfactants, and/or DMSO. Salts may comprise for example, without limitation, NaCl and MgCh.

### Pathogen Signal Enrichment by Selective Denaturation

The concentration of the pathogen or microbe signal in the cell-free nucleic acid fraction is minuscule and requires enrichment of its signal or depletion of human signal in order to detect non-human signal with current sequencing techniques at acceptable cost. Short fragments (<110 base pairs) where pathogen or microbe fragments are present at higher molar fraction can be enriched using approaches such as an electrophoretic-based size selection.

The human fraction in cell-free nucleic acid pool is partially derived from the fragments initially wrapped around the nucleosomal core particle. These fragments are mostly 150-175 base pairs long. The vast majority of the microbial cell-free nucleic acids on the other hand are shorter than 110 base pairs with average lengths about and less than 50 base pairs.

Depletion of the human fragments can be performed by selective denaturation where a controlled amount of heat is introduced into the system that allows the shorter, and thermally less stable cell-free nucleic acids fragments that are enriched for the microbial fraction to denature but leaves intact the longer fragments that are thermally more stable and enriched for human fraction.

In some embodiments, denaturation is used to further enrich pathogen or microbe nucleic acids. In some embodiments, denaturation comprises selective denaturation. In some embodiments, selective denaturation comprises one or more denaturation steps effective for the selection of pathogen or microbe fragments of a certain length and/or GC-content.

In some embodiments, selective denaturation comprises incubation at a selected time. In some embodiments, the selected time comprises about 1 second, about 2 seconds, about 3 seconds, about 4 seconds, about 5 seconds, about 10 seconds, about 15 seconds, about 20 seconds, about 25 seconds, about 30 seconds, about 35 seconds, about 40 seconds, about 45 seconds, about 50 seconds, about 55 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19, minutes about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or about 60 minutes. In some embodiments, incubation occurs at any of the denaturation steps such as, for example, without limitation, following dephosphorylation, preceding 3′-end adapter attachment, and/or during an elution step.

In some embodiments setting the temperature selects for fragments of a certain length and/or GC-content. In some embodiments, fragments of a certain length and/or GC-content comprise fragments having a length less than about 10,000 base pairs, less than about 5,000 base pairs, less than about 4,000 base pairs, less than about 3,000 base pairs, less than about 2,000 base pairs, less than about 1,000 base pairs, less than about 500 base pairs, less than about 450 base pairs, less than about 400 base pairs, less than about 350 base pairs, less than about 300 base pairs, less than about 250 base pairs, less than about 200 base pairs, less than about 180 base pairs, less than about 160 base pairs, less than about 140 base pairs, less than about 120 base pairs, less than about 115 base pairs, less than about 110 base pairs, less than about 105 base pairs, less than about 100 base pairs, less than about 95 base pairs, less than about 90 base pairs, less than about 85 base pairs, less than about 80 base pairs, less than about 75 base pairs, less than about 70 base pairs, less than about 65 base pairs, less than about 60 base pairs, less than about 55 base pairs, less than about 50 base pairs, less than about 45 base pairs, less than about 40 base pairs, less than about 35 base pairs, less than about 30 base pairs, less than about 25 base pairs, less than about 20 base pairs, or less than about 15 base pairs.

### Examples of Nucleic Acid Library Preparation

Disclosed here is a method of preparing a nucleic acid library from a sample comprising a mixture of microbial nucleic acids and human nucleic acids, wherein the method comprises:
a. receiving the sample comprising a mixture of microbial nucleic acids and human nucleic acids, wherein one or more of the human nucleic acids comprise a 5′-end phosphate moiety;
b. attaching a blocking moiety to the 5′-end phosphate moiety to the one or more human nucleic acids of the mixture or degrading the human nucleic acids using a 5′ phosphorylation-specific exonuclease;
c. phosphorylating the microbial nucleic acids of the mixture to produce 5′ phosphorylated microbial nucleic acids; and
d. attaching 5′-end adapters to the 5′ phosphorylated microbial nucleic acids to produce a nucleic acid library enriched for microbial nucleic acids.

In one embodiment, the method further comprises attaching 3′-end adapters to nucleic acids in the mixture of microbial nucleic acids and human nucleic acids. In another embodiment, 5′-end phosphorylation-blocking adapters are attached via the 5′-end phosphate moieties and impede a reaction, resisting ligation, resisting phosphorylation, resisting amplification, or resisting sequencing. In another embodiment, the blocking adapters are splint oligonucleotides that comprise a double-stranded region preferably comprising a uracil base situated not directly connected to the single-stranded region; and a single-stranded region preferably comprising random nucleotides, or a sequence that hybridizes at an end of the human nucleic acids.

In another embodiment the mixture of microbial nucleic acids and human nucleic acids are denatured; the 5′-end adapters are ligated to the 5′-end decoy adapters; and a uracil base or DNA backbone in the 5′-end decoy adapters are cleaved to disconnect the adapters from the human nucleic acids. In another embodiment the human nucleic acids are treated by a 5′ phosphorylation-specific exonuclease, preferably a lambda exonuclease or terminator exonuclease.

In another embodiment, the method further comprises amplifying the microbial nucleic acids attached to 5′-end adapters to enrich for the mcfNA, preferably using primers that hybridize to the 3′-end adapters or the 5′-end adapters, preferably adapters that are double-stranded oligonucleotides, or splint oligonucleotides that comprise a double-stranded region and a single-stranded region, preferably where the single-stranded region comprises random nucleotides or a sequence that hybridizes to an end of the microbial nucleic acids.

In another embodiment, the 5′-end adapters are ligated to the 5′ phosphorylated microbial nucleic acids by a T4 DNA ligase, a SplintR ligase, a PBCV-1 DNA ligase or a Chlorella virus DNA ligase.

In another embodiment the method further comprises denaturing the nucleic acids of the sample (preferably a sample of blood, serum, plasma, bronchial lavage, synovial fluid, bronchoalveolar lavage, or cerebrospinal fluid) to produce denatured nucleic acids prior to addition of the adapters; and results in a 2-fold enrichment of the mcfNA.

Another aspect of the disclosure is a method of preparing a nucleic acid library from a sample comprising nucleic acids, wherein the method comprises:
a. providing a sample comprising 5′-end phosphorylated nucleic acids and 5′-end non-phosphorylated nucleic acids;
b. attaching a decoy 5′-end adapter to the 5′-end phosphorylated nucleic acids to produce nucleic acids attached to the 5′-end adapter, wherein the decoy 5′-end adapter is 5′-end non-phosphorylated and is modified to resist 5′-end phosphorylation;
c. phosphorylating the 5′-end non-phosphorylated nucleic acids with a kinase to produce phosphorylated nucleic acids;
d. attaching a 5′-end adapter to the 5′-end phosphorylated nucleic acids to produce nucleic acids attached to the 5′-end adapters; and
e. amplifying the nucleic acids attached to the 5′-end adapters.

In one embodiment of the method, 3′-end adapters are attached to the nucleic acids that have been modified by 5′-end phosphorylation. In some embodiments, the 3′ end adapters are also attached to nucleic acids that have not been modified by 5′-end non-phosphorylation.

Adapters, full length or partial, may be attached to the nucleic acids in a sample at one or more points during the sample preparation process. In some embodiments, adapters may be attached by ligation, by primer extension, by non-templated extension, by template switching, by the addition of nucleotides to the 3′ terminus of a nucleic acid molecule, by hybridization, by amplification (e.g., PCR) or a combination of any of these reaction types. In some embodiments, adapters are attached by a ligation reaction method using a ligase enzyme that recognizes a particular nucleic acid form. In some embodiments, adapters are attached by a primer extension reaction method using, e.g., a PCR reaction, where the adapter also acts as a primer for a polymerase which acts on a particular nucleic acid form. In some embodiments, adapters are attached with a combination of a non-templated nucleic acid polymerase and primer extension off of non-templated sequences (e.g., template switching or template switching PCR).

Depending on the type of nucleic acid molecule in the sample, the adapter attached can be either double-stranded or single-stranded such that the adapter is compatible with the nucleic acid molecules in the sample. For example, in some embodiments a double-stranded adapter is attached to a double-stranded nucleic acid. In some embodiments, it is desirable to protect adapter ends, for example by adding 5′-end and/or 3′-end protective groups, such as amino modifiers, C3 spacers, dideoxy nucleotides, and/or inverted nucleotides or by providing an adapter that is duplexed on one end (or double-stranded) and single-stranded on the other end. Any combination of protective methods and/or groups set forth herein may be used.

Primer extension reactions can be carried out with a DNA-dependent polymerase, an RNA-dependent polymerase, polymerase with non-templated activity, a reverse transcriptase, or a combination thereof. In some embodiments, the primer extension reaction can be carried out by a DNA or RNA polymerase having strand displacing activity. In some embodiments, the primer extension reaction is carried out by a DNA or RNA polymerase that has non-templated activity. In some other embodiments, the primer extension reaction can be carried out by a DNA or RNA polymerase having strand displacing activity and a DNA or RNA polymerase that has non-templated activity. In some embodiments, primer extension is carried out with a Klenow fragment.

### Adapter Compositions

Particular adapters may be used with the present disclosure. In general, the adapter compositions allow for the detection of different nucleic acid forms in a sample.

Depending on the starting sample type, what nucleic acid(s) are being analyzed, the method, and what detection system is being used, an appropriate adapter can be employed (e.g., particular functional elements or modifications).

In general, an adapter can comprise a polymerase priming sequence, a sequence required to initiate reading of a nucleic acid sequence in sequencing, a sequence required to initiate reading of identifying sequences, and/or one or more identifying sequences (e.g., such as an index, a barcode, a non-templated overhang, a random sequence, unique molecular identifiers, or a combination thereof). For other applications, an adapter can comprise at least one functional element selected from polymerase priming sequence, a sequencing priming sequence, binding sites for amplification primers, a recognition sequence or structural elements required by the sequencing method utilized, one or more identifying sequences, and a label (e.g., radioactive phosphates, biotin, fluorophores, or enzymes). Labels can be added to an adapter if a purification step or particular detection system is desired (e.g., digital PCR, ddPCR, quantitative PCR, microfluidic device, microarray, etcetera).

The adapter may be single-stranded or double-stranded or can have both single-stranded and double-stranded regions. In some embodiments, the adapter comprises an RNA molecule, a DNA molecule, or a molecule that contains both DNA and RNA sections and/or strands, and/or a single strand that has both RNA and DNA components. In some embodiments, a double-stranded adapter may be blunt-ended. In some embodiments, a double-stranded adapter may contain nucleic acid residue overhang(s).

Such nucleic acid residue overhangs (or tails) may be used to mark a molecule as originating from DNA or RNA in the starting sample, particularly when the overhangs are complementary to an overhang sequence deposited by a DNA nucleotidylexotransferase (e.g. TdT), Poly(A) Polymerase, a RT (e.g., SMARTer RT, HIV RT), RNA-dependent polymerase (e.g. RdRP from turnip crinkle virus), and/or a DNA-dependent polymerase (e.g., Bst 2.0 DNA polymerase). For example, the adapter overhang may contain one or more T residues in order to hybridize to one or more overhang residues deposited by a DNA polymerase (e.g., Bst 2.0 DNA polymerase, TdT or the like). Similarly, the adapter overhang may contain one or more C residues in order to hybridize to one or more overhang residues deposited by an RT (e.g., SMARTer RT, reverse transcriptases derived from Moloney Murine Leukemia Virus, or the like). HIV reverse transcriptase and the long terminal repeat retrotransposon also have non-templated activity but may add a different nucleotide other than C.

### Amplification Element

An adapter can comprise an amplification primer that is a primer used to carry out a polymerase chain reaction (PCR). In some embodiments, the amplification primer comprises a random primer. In some embodiments, the amplification primer comprises a template-specific primer. In some embodiments, the amplification primer comprises a primer complementary to a known non-templated overhang known to be added by the polymerase. In some embodiments, the amplification primer comprises a standardized flow cell adapter sequence or a part thereof; standardized flow cell adapter sequences are known in the art and include but are not limited to P5 and P7. In some embodiments, the amplification primer comprises a P5 primer. In some embodiments, the amplification primer comprises a P7 primer. In some embodiments, the amplification primer comprises only part of a P5 or P7 primer. In some embodiments, depending on the method of detection, the amplification primer comprises one or more additional functional elements.

### Identifying Sequence Element

Identifying sequences (e.g., barcode, index, or a combination thereof) can comprise a unique sequence. The identifying sequences can be added to a particular nucleic acid form by the methods provided herein (e.g., ligation, primer extension, amplification, non-templated extension, template switching, template switching PCR or a combination thereof) allowing the identification of each nucleic acid form in a sample or after sequencing. In some embodiments, the identifying sequences may also contain additional functional elements such as primer amplification sites, sequencing priming sites, or sample indexes.

The identifying sequences can be completely scrambled (e.g., randomers of A, C, G, and T for DNA or A, C, G, and U for RNA) or they can have some regions of shared sequence. For example, a shared region on each end may reduce sequence biases in ligation events. In some embodiments, the adapter comprises shared region and the shared region comprises about or at least about 1, 2, 3, 4, 5, 6, 7, 8, 9, 10, 11, 12, 13, 14, 15, 16, 17, 18, 19, or 20 common base pairs.

Combinations of barcodes and/or indexes can be added to increase diversity. For example, barcodes and/or indexes can be used as identifiers for well position in a microtiter plate, array, or the like (e.g., 96 different barcodes for a 96-well plate), and another barcode can be used as an identifier for a plate number (e.g., 24 different barcodes for 24 different plates), giving 96×24=2,304 combinations using 96+24=120 sequences. Using three or more barcodes per sample can further increase achievable diversity.

In some embodiments, the adapter comprises barcodes and/or indexes. In some embodiments, the barcodes and/or indexes are linked to sequencing reads. In some embodiments, particular barcodes and/or indexes may be linked to particular sequencing reads. In some embodiments, particular barcodes and/or indexes may be linked to particular initial sample. In some embodiments, barcodes comprise about 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 200, 250, 300, 350, or 400, 500, or 1000 nucleotides (or base pairs) in length.

### Label Element and Other Components

In some embodiments, the adapter comprises one or more labels. Labels can be added to an adapter when purification is desired or for using particular detection. Examples of labels that can be used with the disclosure include, but are not limited to, any of those known in the art, enzymes such as fluorophores, radioisotopes, stable free radicals, luminescers, such as chemluminescers, bioluminescers, dyes, pigments, enzyme substrates, biotin, digoxigenin, antigens, antibodies, a His-tag, and other labels. One skilled in the art will choose a label that is compatible with the chosen detection method.

### Attaching Adapters & Ligase Enzymes

In some embodiments, attaching comprises using a ligase; in other embodiments attaching comprises using a polymerase. In some embodiments, attaching an adapter comprises attaching an adapter to both DNA and RNA target molecules. When multiple different ligases are used (e.g., a dual ligase system), the ligases may each be specific for a target (e.g., DNA-specific, or RNA-specific). In some embodiments, attaching comprises using a dual ligase system. In some embodiments, the dual ligase system comprises DNA-specific, RNA-specific, and/or ligases that ligate both DNA and RNA templates in any combination.

In some embodiments, the ligase comprises a ligase specific for double-stranded nucleic acids (e.g., dsDNA, dsRNA, RNA/DNA duplex). An example of a ligase specific for double-stranded DNA and DNA/RNA hybrids is T4 DNA ligase. In some embodiments, the ligase is specific for single-stranded nucleic acids (e.g., ssDNA, ssRNA). An example of such ligase is CircLigase II. In some embodiments, the ligase comprises a ligase specific for RNA/DNA duplexes. In some embodiments, the ligase comprises a ligase that is able to work on single-stranded, double-stranded, and/or RNA/DNA nucleic acids in any combination.

Both DNA or/and RNA ligases may be used with the disclosure. Ligases that may be used in the methods provided herein may include, but are not limited to, T4 DNA Ligase, T3 DNA Ligase, T7 DNA Ligase, E. coli DNA Ligase, HiFi Taq DNA Ligase, 9° N™ DNA Ligase, Taq DNA Ligase, SplintR® Ligase (also known as Splint-R ligase or PBCV-1 DNA Ligase or Chiarella virus DNA Ligase), Thermostable 5′ AppDNA/RNA Ligase, T4 RNA Ligase, T4 RNA Ligase 2, T4 RNA Ligase 2 Truncated, T4 RNA Ligase 2 Truncated K227Q, T4 RNA Ligase 2, Truncated KQ, RtcB Ligase, CircLigase II, CircLigase ssDNA Ligase, CircLigase RNA Ligase, Ampligase® Thermostable DNA Ligase, T4 RNA ligase II and its modified or truncated derivatives, or a combination thereof.

In some embodiments, the adapters are attached to nucleic acids, such as, for example, without limitation, a single-stranded RNA, comprising a 5′-end modification such as App (e.g., pre-adenylation). The presence of the 5′ App modification can enable oligonucleotides to act as direct substrates for certain ligases and remove the need for ATP. Adapters to single-stranded RNA can contain a 5′ adenylation (5′ App) modification and/or an RNA-identifying code.

Alternatively, or additionally, DNA and RNA in a sample can be specifically marked during an adapter attachment step. In some embodiments the adapter attachment step may involve template switching or ligation. In some embodiments, the ligase comprises a ligase specific for one type of nucleic acids. For example, a DNA-specific ligase may be used so that adapters are only ligated to the DNA molecules in the sample. In another example, an RNA-specific ligase may be used so that adapters are only ligated to the RNA molecules in the sample. In some embodiments, ligation comprises successive ligation with a first ligase specific to one type of nucleic acid and a second ligase not discriminating between nucleic acids types. For example, successive ligation first with a DNA-specific ligase (e.g., CircLigase ssDNA ligase) followed by a ligase that can act on a DNA or RNA template (e.g., CircLigase II) may be used. Sequential or concurrent first adapter attachment and/or sequential or concurrent second adapter attachment may provide the ability to distinguish between chemical forms of nucleic acids (e.g., DNA and RNA). The choice of ligation method may depend on the ligase specificities and reaction conditions for each ligase used.

In some embodiments, the ligase comprises ligase selected with an appropriate profile of contaminating nucleic acids so that the profile deters sufficiently from an expected signal of interest (e.g., endogenous microbe signal in cell-free nucleic acid pool) in order to recognize and filter contamination signal originating from ligase. In some embodiments, an appropriate profile of contaminating components (e.g., buffers, buffer components, oligonucleotides, enzymes, water, beads, etc.) is selected so that the profile deters sufficiently from an expected signal of interest in order to recognize and filter contamination signal originating from components.

### Successive Mode of Attachment

The methods provided by the present disclosure can be applied in a successive mode, that is more than one enzymatic step can be applied at separate steps in the process. In some embodiments when successive ligation is used, a wash step can be performed between the two ligation reactions to remove the first ligase and excess adapters. For example, successive ligation can be used in the first adapter ligation step. Biotinylated first adapters with a code for DNA (e.g., 1a adapters) can be added to the sample nucleic acids and ligated to ssDNA using a DNA ligase. Ligation products can be immobilized on streptavidin beads.

Excess 1a adapters can be washed off First adapters with a code for RNA (1b adapters) can be added and ligated to ssRNA using an RNA ligase.

In general, for each ligation step (e.g., first ligation, second ligation, pre-denaturation ligation), a single general adapter or specific adapters can be used. In some embodiments, a single adapter is added to all nucleic acids in a ligation step. In some embodiments, a single adapter is added to a specific group of nucleic acids (e.g., only single-stranded or only double-stranded for a pre-denaturation ligation) in a ligation step. In some embodiments, different adapters can be added to specific groups of nucleic acids (e.g., ssDNA, ssRNA, dsDNA, or dsRNA). In some embodiments, selectivity can be achieved through enzymatic selectivity with a wash step in between sequential enzymatic steps to remove excess unadapted adapters. In some embodiments, selectivity can be achieved through sequence-specific hybridization to different overhangs added by polymerases in the primer extension step. In some embodiments, a polymerase attaches the adapter sequence with the splint wherein the splint binds anywhere on the original strand and the polymerase performs the primer extension reaction, thus doing multiple steps concurrently.

### Fragmentation & End Modification

In some embodiments, the methods do not include fragmenting the nucleic acids, such as, in application with low quality samples or samples containing short fragments such as certain samples containing cell-free nucleic acids.

In some embodiments, nucleic acids are fragmented. Fragmenting of the nucleic acids may be performed by e.g., mechanical shearing, passing the sample through a syringe, sonication, heat treatment, any other method in the art, or a combination thereof. In some embodiments, shearing may be performed by mechanical shearing (e.g., ultrasound, hydrodynamic shearing forces), enzymatic shearing (e.g., endonuclease), thermal fragmentation (e.g., incubation at high temperatures), chemical fragmentation (e.g., alkaline solutions, divalent ions). In some embodiments, fragmenting can be performed by using an enzyme, including a nuclease, or a transposase. Nucleases used for fragmenting comprise restriction endonucleases, homing endonucleases, nicking endonucleases, high fidelity restriction enzymes, or any enzyme disclosed herein.

The ends of dsDNA fragments can be polished (e.g., blunt-ended). The ends of DNA fragments can be polished by treatment with a polymerase. Polishing can involve removal of 3′ overhangs, fill-in of 5′ overhangs, or a combination thereof. The polymerase can be a proofreading polymerase (e.g., comprising 3′ to 5′ exonuclease activity). The proofreading polymerase can be, e.g., a T4 DNA polymerase, Pol 1 Klenow fragment, or Pfu polymerase. Polishing can comprise removal of damaged nucleotides (e.g., abasic sites), using any means known in the art.

Reduction of Adapter Dimers and Adapter by-Products

Some methods may produce adapter dimers and adapter-derived by-products. Adapter dimers and adapter-derived by-products are two classes of unwanted products of a single-stranded library protocol that are generated by two distinct mechanisms.

For example, the single-stranded nucleic acid library protocol developed by Gansauge et al. generates high concentration of adapter dimers and adapter-derived by-products, especially with input samples characterized by low nucleic acid concentration (See, Gansauge, MT and Meyer, M., Single-stranded DNA library preparation for the sequencing of ancient or damaged DNA, Nat Protoc. 2013 April; 8(4):737-48 and Gansauge MT, Gerber T, Glocke I, Korlevic P, Lippik L, Nagel S, Riehl LM, Schmidt A, and Meyer M., Single-stranded DNA library preparation from highly degraded DNA using T4 DNA ligase, Nucleic Acids Res. 2017 Jun. 2; 45(10), each of which is incorporated by reference in their entirety herein, including any drawings).

One way to decrease adapter-derived by-products according to an embodiment of the disclosure comprises using an RNA splint oligonucleotide. In some embodiments, attaching a 3′-end adapter to the denatured nucleic acids and/or single-stranded nucleic acids comprises attaching said adapter with a splint oligonucleotide. In some embodiments, the splint oligonucleotide comprises a DNA splint oligonucleotide. In some embodiments, the splint oligonucleotide comprises an RNA splint oligonucleotide or a partial RNA splint oligonucleotide. In some embodiments, attaching a 3′-end adapter to the denatured nucleic acids and/or single-stranded nucleic acids comprises ligating with a Splint-R ligase. In some embodiments, attaching a 3′-end adapter to the denatured nucleic acids further comprises adding an RNase inhibitor. In some embodiments, an adapter is attached through a primer extension reaction performed with a polymerase comprising DNA-dependent RNA-dependent polymerase, or a polymerase having non-templated activity.

Some embodiments comprise preventing ligation of the 5′-end adapter to a complement synthesized during the primer extension reaction (set forth above) during a second ligation step. Some embodiments comprise preventing ligation of the 5′-end adapter to an adapter-derived side product. Digoxigenin may be introduced to the 5′-end of the splint oligo and an anti-digoxigenin antibody may be added to the bead-binding buffer during immobilization of adapted products. An anti-digoxigenin antibody can be added at any point prior to the second ligation. This will produce a bulky moiety at the 5′-end of any splint oligo attached to the biotinylated 3′-end adapter. This moiety will reduce the ability of T4 DNA ligase in a second ligation step to ligate 5′-end adapter to splint oligo hybrid rendering it un-amplifiable in the final PCR step. It may also reduce the efficiency of primer-extension.

Some embodiments comprise adding an antibody, such as an anti-digoxigenin antibody. In some embodiments, the anti-digoxigenin antibody is added after the 3′-end adapter is attached to the denatured nucleic acids and before a 5′-end adapter is attached. Some embodiments further comprise using beads comprising anti-digoxigenin antibody. Beads may be removed by, for example, without limitation, pelleting on a magnet. For example, an anti-digoxigenin antibody-coated magnetic bead can be added to deplete digoxiginated splint oligos as well as any unhybridized digoxiginated splint oligos. This can be followed by streptavidin-coated magnetic bead. In some embodiments, the anti-digoxigenin antibody is added during a separation step, annealing step, primary extension step, or second ligation step.

### Detection, Abundance, Bias, and Contamination

Libraries of the disclosure may be used to distinguish populations or mixtures of nucleic acids, e.g. populations of cfDNA. Libraries of the disclosure may be used for detection. Non-limiting examples of detection which can be used with the nucleic acid libraries set forth herein include various forms of sequencing, qPCR, ddPCR, microfluidic device, or microarray.

### Process Control Molecules

One or more process control molecules may be added to a sample (e.g., initial sample, raw sample, etc.) for various reasons, for example, without limitation, in order to facilitate accuracy in the process of distinguishing one population of nucleic acids e.g. cfDNA from another (or multiple populations of nucleic acids from each other). In some cases, process control molecules may have special features such as specific sequences, lengths, GC content, degrees of degeneracy, degrees of diversity, secondary, tertiary, and quaternary structure, and/or known starting concentrations. In another embodiment, process control molecules may be used for normalizing signal in sample (e.g., an initial sample) in order to account for variations in sample processing. In some embodiments, process control molecules can be added during the library process itself, e.g., without limitation, dephosphorylation controls may be added before and after dephosphorylation or attachment control before and/or after the 3′-end adapter attachment step. Process control molecules may include, but are not limited to, ID Spike(s), Spanks, and/or Sparks or GC Spike-in Panel molecules. In some embodiments, process control molecules may comprise ID Spike(s), Spanks, and/or Sparks or GC Spike-in Panel molecules.

ID Spike(s) refers to identification spikes used for sample identification tracking, distinguishing different populations of nucleic acids, e.g., distinguishing quantitatively or qualitatively, cross-contamination detection, reagent tracking, and/or reagent lot tracking (See, for example, U.S. Pat. No. 9,976,181). Spanks are degenerate pools of nucleic acids, or pools of nucleic acids with diverse sequences, used for diversity assessment and abundance calculation (id.). Sparks, “GC Spike-in Panel,” or “GC dSPARKS” are size or length markers which may be used for abundance, normalization, development and/or analysis purposes, process performance monitoring, and other purposes (id.).

Process control molecules may additionally include molecules designed to monitor individual steps of the process. Process control molecules may additionally include dephosphorylation control molecules, denaturation control molecules, ligation control molecules, and/or control molecules for non-templated extension or template switching. Partially or fully phosphorylated control molecules (i.e., phosphorylated 5′-end and/or 3′-ends of the control nucleic acids), control molecules with adapter sequences pre-attached (i.e., an example of a control molecule that is added after 3′-end adapter attachment step) may be added during the library process itself, e.g., dephosphorylation control post dephosphorylation step or adapter attachment control post 3′-end adapter attachment step. In some embodiments, process control molecules comprise dephosphorylation control molecules, denaturation control molecules, and/or ligation control molecules.

An example of a ligation process control molecule that can be added after a 3′-end ligation step is ATGACGCGCTTTCAAGCGTGGCGAGTATGTGAACCAAGGCTTCGGACAGGAGATCGGAAG/iSpC3/iSpC3/iSpC3/iSpC3/iSpC3/iSpC3/iSpC3/iSpC3/iSpC3/iSpC3/3BioTEG/(SEQ ID NO: 1).

Exemplary denaturation control molecules: Two nucleic acid sequences, the first one added in single-stranded form (e.g., ACTATATACTTAGGTTTGATCTCGCCCCGAGAACTGTAAACCTCAACATT (SEQ ID NO: 2)), and the second one, a close sequence relative to the first one, but added in a double-stranded form (e.g., TGAAATATCTTAGGTTTGATCTCGCCCCGAGAACTGTAAACCTCAACATT (SEQ ID NO: 3)).

Examples of dephosphorylation control molecules include, without limitation: GGCCTCGCGGAGGCATGCGTCATGCTAGCGTGCGGGGTACTCTTGCTATC (SEQ ID NO: 4); GAGAATTATTCGGGGGCAGTGACAACCAACATCTCGGGTCCTGCCCAACC-3′Phosph (SEQ ID NO: 5); 5′Phosph-GGTCTACACGCTAATATAGCGAATCACCGAGAACCCGGCGCCACGCAATG-3′Phosph (SEQ ID NO: 6); and 5′Phosph-GAACGTCCTTAACTCCGGCAGGCAATTAAAGGGAACGTATGTATAACGCA (SEQ ID NO: 7),

where “5′Phosph” and “3′Phosph” indicate that the 5′-end and 3′-end of the control molecule is phosphorylated, respectively. The dephosphorylation control molecules are represented in single-stranded form, but the dephosphorylation control molecules may be double-stranded, RNA and/or any other form of modified nucleic acids.

### Sequencing

Some embodiments comprise sequencing the nucleic acid libraries of the current disclosure to generate sequencing information. Some embodiments further comprise a computer comprising software that performs bioinformatics analysis on the sequence information. Bioinformatics analysis comprises without limitation, assembling sequence data, detecting and quantifying genetic variants in a sample, including germline variants and somatic cell variants (e.g., a genetic variation associated with cancer or a pre-cancerous condition, a genetic variation associated with infection), detecting species or strain of microbes, detecting microbes at a certain taxonomic level (e.g., order, family, genus, species, strain), detecting presence and measuring the abundance of microbe nucleic acids, detecting presence and measuring the abundance of therapeutic nucleic acids, detecting site of infection, detecting risk of transplant rejection, detecting state of infection, and/or detecting potential for drug resistance.

Sequencing may be used to analyze nucleic acids; particularly different forms of nucleic acids present in the same sample. Such analytical methods include sequencing the nucleic acids as well as bioinformatics analysis of the sequencing results. Sequencing results may be analyzed to obtain various types of information including genomic and RNA expression. Generally, analyses provided herein allow for simultaneous analysis of DNA and RNA in a sample, as well as both single- and double-stranded nucleic acids in a sample.

In some embodiments, the analysis detects both DNA and RNA yet does not distinguish between the two. In some embodiments, the analysis detects both DNA and RNA (or double- and single-stranded nucleic acids) and also identifies whether the originating molecules are DNA, RNA, ssDNA, dsDNA, ssRNA, dsRNA, or any combination of the molecules. Often, distinguishing is accomplished by detecting markers added by using a combination of adapters specific to a molecule type of interest and/or appropriate enzyme that facilitate and enhance discrimination between different nucleic acid types (RNA vs DNA, single- vs double-stranded).

Sequencing may be by any method known in the art. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (e.g., Ion Torrent sequencing), nanopore sequencing, pyrosequencing (454), sequencing by synthesis, sequencing by ligation (SOLiD sequencing), sequencing by electron microscopy, dideoxy sequencing reactions (Sanger method), massively parallel sequencing, polony sequencing, and DNA nanoball sequencing. The term “Next Generation Sequencing (NGS)” herein refers to sequencing methods that allow for massively parallel sequencing of nucleic acid molecules during which a plurality, e.g., millions, of nucleic acid fragments from a single sample or from multiple different samples are sequenced simultaneously. Non-limiting examples of NGS include sequencing-by-synthesis, sequencing-by-ligation, real-time sequencing, and nanopore sequencing. In some embodiments, sequencing involves hybridizing a primer to the template to form a template/primer duplex, contacting the duplex with a polymerase enzyme in the presence of detectably labeled or unlabeled nucleotides under conditions that permit the polymerase to add labeled or unlabeled nucleotides to the primer in a template-dependent manner, detecting a signal from the incorporated labeled nucleotide or detecting a signal resulting from the process of incorporating labeled or unlabeled nucleotide (e.g., proton release), and sequentially repeating the contacting and/or detecting steps at least once, wherein sequential detection of incorporated labeled or unlabeled nucleotide determines the sequence of the nucleic acid.

Exemplary detectable labels include radiolabels, fluorescent labels, protein labels, dye labels, enzymatic labels, etc. In some embodiments, the detectable label may be an optically detectable label, such as a fluorescent label. Exemplary fluorescent labels include cyanine, rhodamine, fluorescein, coumarin, BODIPY, alexa, or conjugated multi-dyes.

In some embodiments, the sequencing comprises obtaining paired end reads. In some embodiments, the sequencing comprises obtaining consensus reads.

The accuracy or average accuracy of the sequence information may be greater than about 80%, about 90%, about 95%, about 99%, about 99.98%, or about 99.99%. The sequence accuracy or average accuracy may be greater than about 95% or about 99%. The sequence coverage may be greater than about 0.00001-fold, 0.0001-fold, 0.001-fold, about 0.01-fold, about 0.1-fold, about 0.5-fold, about 0.7-fold, or about 0.9-fold. The sequence coverage may be less than about 200,000-fold, about 100,000-fold, about 10,000-fold, about 1,000-fold, or about 500-fold.

In some embodiments, the sequence information obtained per nucleic acid template is more than about 10 base pairs, about 15 base pairs, about 20 base pairs, about 50 base pairs, about 100 base pairs, or about 200 base pairs. The sequence information may be obtained in less than 1 month, 2 weeks, 1 week, 2 days, 1 day, 14 hours, 10 hours, 3 hours, 1 hour, 30 minutes, 10 minutes, or 5 minutes.

Although the Examples (below) use specific sequences (see Table 1 in Example 1) for certain sequencing systems, e.g., Illumina systems, it will be understood that the reference to these sequences is for illustration purposes only. For example, the methods described herein are not exhaustive and may be configured for use with other sequencing systems incorporating specific priming, attachment, index, and other operational sequences used in those systems, e.g., systems available from Ion Torrent, Oxford Nanopore, Genia Technologies, Pacific Biosciences, Complete Genomics, and the like.

### Applications

Nucleic acid libraries set forth herein can be used for a variety of applications including personalized medicine. Specifically, the nucleic acid libraries can be used to detect, monitor, diagnose, prognose, guide treatment, or predict the risk of disease. Exemplary applications are provided below.

### A. Cancer

The nucleic acid libraries can be used for detecting cancer in a subject or for cancer diagnosis. Initial samples may be either somatic, germline, or a combination thereof. Initial samples can be from bodily fluids, organs, cells, blood, tissue, or any bodily sample known to harbor the cancer mutation. In some cases, a cancer mutation is present within cell-free nucleic acids (e.g., circulating cell-free nucleic acids) or within a circulating tumor cell. In some embodiments, the nucleic acid library from the initial sample is sequenced and assessed for the detection or diagnosis of cancer in a subject.

### B. Fetal Health

The nucleic acid libraries can be used for distinguishing mixed populations of nucleic acids, e.g., cfDNA or can be used for detection, diagnosis, or prognosis of fetal health (e.g., a IVF embryo or a fetus) in a subject. In some embodiments, the nucleic acid libraries can be used to determine or assess the risk of infection status of an embryo or fetus. In some embodiments, nucleic acid libraries can be used for the assessment of chromosomal aberrations, e.g., aneuploidy. In some cases, the nucleic libraries can be used to detect an inherited condition including but not limited to, autosomal-recessive, dominant, X-linked, or SNP-based genetic conditions in a subject. In some cases, the nucleic libraries are used to detect germ-line genetic aberrations. In some cases, the nucleic libraries are used to detect somatic or acquired genetic aberrations. In some embodiments, the nucleic acid library from the sample (e.g., initial sample) is sequenced and assessed to distinguish populations of nucleic acids or for the detection, diagnosis, or prognosis of fetal health in a subject.

### C. Organ Transplant

The nucleic acid libraries can be used to distinguish populations of nucleic acids or for the detection, diagnosis, or prognosis of organ transplant acceptance or rejection in a subject. Transplant rejection occurs when transplanted tissue is rejected by a recipient's immune system. Incompatibility across key HLA alleles has traditionally been considered the main factor influencing rejection in stem cell therapies, adoptive cell therapies, immunotherapies or in solid organ transplants. The effect of specific HLA mismatches in kidney transplantation are known in the art. Even in HLA identically matched kidney transplantation, some degree of rejection is still evident. Non-HLA or minor histocompatibility antigens (mHAs) resulting from a range of functional polymorphisms in the genome have been suggested to be capable of inducing strong cellular immune responses. In some embodiments, the nucleic acid library from the initial sample is sequenced and assessed to distinguish populations of nucleic acids or for the detection, diagnosis, or prognosis of organ transplant acceptance or rejection in a subject.

### D. Microbe or Pathogen Detection and Quantification

The methods can be used for to distinguish populations of nucleic acids or for detecting a pathogenic infection in a subject, as well as the symbiotic presence of microbes in a host, such as commensals and a normal host microbiome. In some embodiments, the methods may provide a more comprehensive view of the state and diversity of the infection or symbiotic microbes in a subject. For example, the identification of both RNA and DNA in a sample may be useful to detect RNA and DNA type viruses, or to detect bacterial, protist, parasitic or fungal genomic DNA and/or gene expression products, e.g., mRNA. Such process may also be able to differentiate between latent infection (e.g., which might be indicated by the presence of integrated retroviral DNA) versus active infection (e.g., which might be indicated by the presence of viral RNA from intact viral particles). Such processes may also be able to detect drug resistance and/or the origin of infection. Such processes may also be used to analyze host response. Such analyses may include analysis of cell-free, circulating nucleic acids, e.g., for microbial or viral infection identification.

In an infected sample, nucleic acid forms within a given sample may include a variety of different structural forms and hybrids of those forms, including DNA and RNA, single and double-stranded forms of these, and structured and unstructured forms of these. By way of example, in the case of pathogen identification, it will be appreciated that pathogenic organisms may include a variety of chemical and/or structural forms of nucleic acids that may be used in their identification. As another example, pathogenic organisms may also include chemical modifications of DNA and RNA, some which may confer pathogenicity or make a pathogenic microbe harmless.

In some embodiments, the nucleic acid library from the initial sample is sequenced and assessed for detecting a pathogenic infection in a subject, as well as the symbiotic presence of microbes in a host, such as commensals and a normal host microbiome.

### Kits and Systems

In some cases, this disclosure provides kits and systems. The kits and/or systems may be used, for example, to enrich for a particular population of nucleic acids (e.g., microbial cell-free DNA) present in a mixture. In some cases, the kits and/or systems are used to identify a particular population of nucleic acids (e.g., microbial cell-free DNA) present in a mixture (e.g., a mixture of human and microbial cell-free DNA).

In some cases, this disclosure provides a kit comprising a 5′ splint oligonucleotide and a 5′ blocking splint oligonucleotide. In some cases, the 5′ splint oligonucleotide comprises (i) a double-stranded adapter region comprising a first adapter strand (similar to the 5′ ligation oligo complement 122 depicted in FIG. 1B) and a second adapter strand (similar to the 5′ ligation oligo 121 depicted in FIG. 1B) and (ii) a single-stranded region attached to the first strand of the double-stranded adapter region. In some cases, the single-stranded region (or overhang region) comprises random or degenerate nucleotides. In some cases, the 5′ splint oligonucleotide comprises a splint region (e.g., 122+123 in FIG. 1B) that contains the random region and the first strand of the double-stranded adapter region (or 5′ ligation oligo complement). In some cases, the splint region comprises one or more uracil bases. In some cases, the second adapter strand (or 5′ ligation oligo, 121) does not comprise uracil bases.

In some cases, the 5′ blocking splint oligonucleotide comprises (i) a double-stranded blocking adapter region comprising a first blocking adapter strand (similar to the 5′ dummy oligo complement 112 depicted in FIG. 1B) and a second blocking adapter strand (similar to the 5′ dummy oligo 111 depicted in FIG. 1B) and (ii) a single-stranded region attached to the first strand of the double-stranded blocking adapter region, wherein the second blocking adapter strand has a 5′ terminus that is resistant (or not amenable to) ligation or phosphorylation. In some cases, the single-stranded region (or overhang region, e.g., 113) comprises random or degenerate nucleotides. In some cases, the single-stranded region of the 5′ splint oligonucleotide, the 5′ splint blocking oligonucleotide, the 3′ splint oligonucleotide, or any combination thereof, comprises a random N-mer region.

In some instances, the 5′ terminus of the second blocking adapter strand (e.g., 5′ dummy oligo 111) is not phosphorylated and is resistant to being phosphorylated by a kinase (e.g., PNK). In some cases, the 5′ blocking splint oligo comprises a splint region (e.g., 114). In some cases, the splint region comprises uracil bases. In some cases, the second blocking adapter strand (e.g., 5′ dummy oligo 111) comprises uracil bases. In some cases, the second blocking adapter strand (e.g., 5′ dummy oligo 111) does not comprise uracil bases. In some cases, at least one uracil base is present in the single-stranded random region, the first strand of the double-stranded blocking adapter region, or both the single-stranded random region and the first strand of the double-stranded blocking adapter region. In some cases, at least one uracil base is present in the second strand of the double-stranded blocking adapter region.

In some cases, the kit further comprises a 3′ splint oligonucleotide comprising (i) a double-stranded adapter region comprising a first adapter strand and a second adapter strand and (ii) a single-stranded random region attached to the first strand of the double-stranded adapter region. In some cases, the single-stranded region (or overhang region) comprises random or degenerate nucleotides. In some cases, the 3′ splint oligonucleotide does not comprise a uracil base. In some cases, the 3′ splint comprises a splint region that comprises at least one uracil base.

In some instances, the kit further comprises a kinase. For example, the kit may comprise a PNK kinase. In some cases, the kit comprises a ligase (e.g., T4 ligase). In some cases, the kit further comprises a uracil DNA glycosylase (UDG). In some cases, the kit further comprises an endonuclease. In some cases, the endonuclease is DNA glycosylase-lyase Endonuclease VIII.

In some cases, the kit comprises one or more process control molecules. For example, the kit may comprise SPANKs, SPARKs, ID SPIKEs, or other process control molecules described herein.

In some cases, the oligonucleotides and/or reagents in a kit provided herein are present in a buffer. In some cases, they are lyophilized.

The kit or system may further comprise a software package for data analysis, which may include reference profiles for comparison with the test profile from a clinical sample, and in particular may include reference databases.

In some cases, the 5′ splint oligonucleotide, the 5′ blocking splint oligonucleotide, and/or the 3′ splint oligonucleotide are packaged in separate vials or containers. In some cases, one or more of the oligonucleotides are present in the same vial or container.

In some cases, the kit (or kits) may include instructions on how to use the kit; the instructions may be recorded on any suitable recording medium, e.g. paper, electronic format, etc. The instructions of FIGS. 1A and 1B may be present in the kit as a package insert, in the labeling of the container of the kit, or kit components thereof (i.e., associated with the packaging or sub packaging), etc. In some embodiments, the instructions can be obtained virtually or remotely and may be downloadable or printable e.g. via the internet, email, fax, etcetera. process. The random region in the splint oligo which may comprise one or more uracil bases, is recognizable by USER enzyme. The kit may comprise one or more USER enzymes and reagents to complete a USER digestion reaction including any positive control for this reaction.

The kit may comprise reagents and steps to isolate the different population of nucleic acids. The kit may comprise specific instructions regarding use of each of the oligos, reagents and handling of such. The kit may provide instructions to direct the purification or isolation, purification of nucleic acids herein, e.g. the purification of cfDNA or sample DNA and further instructions on how to proceed with any amplification reactions, sequencing reactions etcetera including any analytical procedures.

Such kits or systems may also include information, such as scientific literature references, package insert materials, clinical trial results, and/or summaries of these and the like. Such kits or systems may also include instructions to access a database. Kits or systems described herein can be provided, marketed and/or promoted to health providers, including physicians, nurses, pharmacists, formulary officials, and the like. Kits or system may also be marketed directly to the consumer.

The kit or system can further comprise an apparatus for detection and/or computer control systems with machine-executable instructions to implement the methods. In some embodiments, computer control systems are further programmed for conducting genetic analysis. Detection systems that can be used including, but are not limited to, sequencing, digital PCR, ddPCR, quantitative PCR (e.g., real-time PCR), or by a microfluidic device, microarray, or the like.

### Hardware Systems

A kit or system can include a nucleic acid sequencer (e.g., DNA sequencer, RNA sequencer) for generating DNA or RNA sequence information. The kit or system may further include a computer comprising software that performs bioinformatics analysis on the DNA or RNA sequence information. Bioinformatics analysis can include, without limitation, assembling sequence data, detecting and quantifying genetic variants in a sample, including germline variants and somatic cell variants (e.g., a genetic variation associated with cancer or pre-cancerous condition, a genetic variation associated with infection), or used to distinguish populations of nucleic acids or in detecting the presence and measuring the abundance of microbe nucleic acids, detecting site of infection, detecting the state of infection, detecting the risk of organ rejection in a transplant patient, and/or detecting potential for drug resistance. One skilled in the art would appreciate other bioinformatics analysis.

Sequencing data may be used to determine genetic sequence information, such as, for example, without limitation, species information, ploidy states, the identity of one or more genetic variants, as well as a quantitative measure of the variants, including relative and absolute relative measures. The sequencing may be unbiased and may involve sequencing all, substantially all, or some (e.g., greater than about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 20%, about 30%, about 40%, about 50%, about 60%, about 70%, about 80%, or about 90%) of the nucleic acids in a sample. Sequencing can be selective, e.g., directed to portions of the genome of interest. For example, many select genes (and mutant forms of these genes) are known to be associated with antibiotic resistance, drug resistance, genetic disorders, and various cancers. Many select genes (and mutant forms of these genes) associated with antibiotic resistance, drug resistance, genetic disorders, and various cancers are also known to be amplified. Sequencing of the select genes, portions of genes, or non-genes along with other genes or sequences may suffice for the analysis desired. Polynucleotides mapping to specific loci in the genome that are the subject of interest can be isolated for sequencing by, for example, sequence capture or site-specific amplification.

### Computer Control Systems

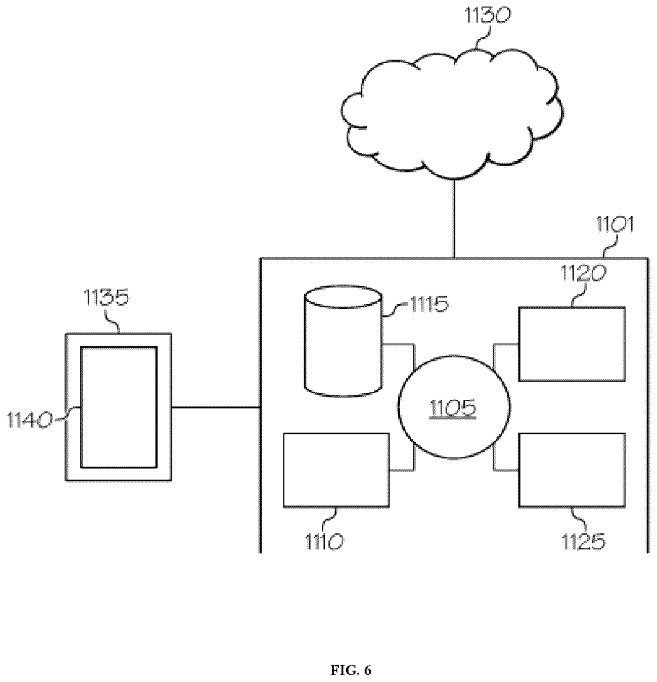

The kit or system can also include computer control systems with machine-executable instructions to implement the methods. FIG. 6 shows a computer system 1101 that is programmed or otherwise configured to implement methods of the present disclosure The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters.

The memory 1110, storage unit 1115, interface 1120, and peripheral devices 1125 are in communication with the CPU 1105 through a communication bus (solid lines), such as a motherboard. The storage unit 1115 can be a data storage unit (or data repository) for storing data.

The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some embodiments is a telecommunication and/or data network. The network 1130 can include one or more computer servers, which can enable distributed computing, such as cloud computing.

The network 1130, in some embodiments with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some embodiments, the circuit is an application specific integrated circuit (ASIC).

The storage unit 1115 can store files, such as drivers, libraries, and saved programs. The storage unit 1115 can store user data, e.g., user preferences and user programs. The computer system 1101 in some embodiments can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., APPLE® iPad, SAMSUNG® Galaxy Tab), telephones, Smart phones (e.g., APPLE® iPhone, Android-enabled device, BLACKBERRY®), or personal digital assistants. The user can access the computer system 1101 via the network 1130.

The kit or system can be implemented by way of machine (e.g., computer processor) executable code stored on an electronic storage location of the computer system 1101, such as, for example, on the memory 1110 or electronic storage unit 1115. The machine executable or machine-readable code can be provided in the form of software. During use, the code can be executed by the processor 1105. In some embodiments, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processor adapted to execute the code or can be compiled during runtime. The code can be supplied in a programming language that can be selected to enable the code to execute in a pre-compiled or as-compiled fashion.

Parts of the kits and systems, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, an output of a report, which may include a diagnosis of a subject or a therapeutic intervention for the subject. Examples of UI's include, without limitation, a graphical user interface (GUI) and web-based user interface. The analysis can be provided as a report. The report may be provided to a subject, to a health care professional, a lab-worker, or other individual.

Methods and systems of the present disclosure can be implemented by way of one or more algorithms. An algorithm can be implemented by way of software upon execution by the central processing unit 1105. The algorithm can, for example, facilitate the enrichment, sequencing and/or detection of pathogen or microbe or other target nucleic acids.

Information about a patient or subject can be entered into a computer system, for example, patient background, patient medical history, or medical scans. The computer system can be used to analyze results from a method described herein, report results to a patient or doctor, or come up with a treatment plan.

### Definitions

Unless defined otherwise, all technical and scientific terms used herein have the meaning commonly understood by a person skilled in the art to which this disclosure belongs.

“A,” “an,” and “the”, as used herein, can include plural references unless expressly and unequivocally limited to one reference.

As used herein, the term “or” is used to refer to a nonexclusive “or”; as such, “A or B” includes “A but not B,” “B but not A,” and “A and B,” unless otherwise indicated.

As used throughout the specification herein, the term “about” when referring to a number or a numerical range means that the number or numerical range referred to is an approximation within experimental variability (or within statistical experimental error), and the number or numerical range may vary from, for example, from 10% to 25% of the stated number or numerical range. In examples, the term “about” refers to +20% of a stated number or value. In other examples, the departure from equimolarity in the case of mixes intended to be equimolar, such as but not limited to, some control molecules in the spike-in mixes, is no more than a ten-fold disparity, an eight-fold disparity, a six-fold disparity, a four-fold disparity or a two-fold disparity.

As used herein, “abundance” refers to the quantity of something, such as, for example, the quantity or number of molecules, such as nucleic acids. As used herein, “relative abundance” is the abundance of a molecule or molecules of interest per abundance of a reference molecule or molecules of interest. For example, relative abundance of target nucleic acid molecules (e.g., pathogen nucleic acid molecules, fetal nucleic acid molecules, tumor-derived nucleic acid molecules, etc.) refers to abundance per reference nucleic acids (e.g., human nucleic acids, synthetic nucleic acid added to the sample, etc.). As used herein, “absolute abundance” is the abundance of molecules per a defined unit of initial sample or sample quantity. For example, absolute abundance of target nucleic acid molecules (e.g., microbe or pathogen molecules, fetal nucleic acid molecules, tumor-derived nucleic molecules, etc.) refers to the abundance per defined unit of sample quantity (e.g., sample volume, sample mass etc.).

As used herein, “antibody” refers to a type of immunoglobulin molecule and is used in the broadest sense to include intact antibodies as well as antibody fragments.

As used herein, antibodies comprise at least one antigen-binding domain. For example, an antibody as described herein may have an antigen binding domain or antigen binding region, the antigen binding domain or antigen binding region being specific for an antigen. In some embodiments, the antigen is a bulky moiety, such as digoxigenin.

As used herein, “adapter” or “portions of an adapter” refers to a chemically synthesized, single-stranded, or double-stranded oligonucleotide that can be attached, e.g., covalently (e.g., ligation) or non-covalently (e.g., hybridization), to the ends of nucleic acid molecules, such as DNA or RNA molecules. Adapter may refer to either a full-length adapter or a portion of the adapter, e.g., partial adapters may be attached in some embodiments before the full-lengths are introduced by e.g., indexing primers in amplification steps. 3′-end adapters and 5′-end adapters may be full-length or a portion of an adapter sequence that are attached to the opposite ends of a target nucleic acid, a copy of a target nucleic acid, or a target nucleic acid complement. 3′-end adapters and 5′-end adapters sequences end up being attached to the opposite ends of e.g., a template that can be sequenced that comprises target nucleic acid, a copy of a target nucleic acid, and/or a target nucleic acid complement. The 3′-end adapter and 5′-end adapter sequences can be the same or they can be different. Adapter sequences may be of any length.

As used herein, “bulky moiety” refers to a molecule that takes up more space than is conventionally required or a molecule that forms a complex that takes up more space than is conventionally required. A bulky moiety may comprise any reactive group capable of forming covalent, non-covalent, or coordinating chemical bonds. In some embodiments, the bulky moiety comprises one or more azide groups and products of reactions with azide groups, one or more small molecules, one or more polyhistidine tags, one or more antigens, and/or one or more proteins. In some embodiments, the bulky moiety comprises digoxigenin. In some embodiments, the splint oligonucleotide with a bulky moiety comprises 5Sp9/A/iDiGN/A/iSp9/CTTCCGATCTNNNNNN/3AmMO, using designations for oligomer modifications adopted from designation convention used by IDT (Coralville, IA; idtdna website). A bulky moiety for example, can include a functional group that is sterically hindering and can prevent certain enzymatic or chemical reactions from occurring. A bulky group can block a position. A bulky group can also affect a molecule's shape and reactivity and so prevent a reaction from occurring through the steric hindrance. Moieties attached to the groups by covalent, non-covalent or coordinating bonds may provide the bulkiness of the groups. For example, a bulky molecule such as a protein or polymer can be attached covalently to an azide group; a bulky entity such as a bead, protein or polymer can be attached to a histag through coordinating bonds using Ni ions; or an antigen antibody can be attached to an antigen attached to an adapter such as an anti-digoxigenin antibody can be attached to digoxigenin. Examples of bulky moieties may also include, but are not limited to, complexes between any of the molecules disclosed herein and their respective binding and reaction partners including, for example, without limitation, complexes between digoxigenin and an anti-digoxigenin antibody; polyhistidine tag and a Ni-NTA-containing polymer; a protein and a binding partner; an azide group and a covalently bound large molecule; and the biotin and streptavidin complex. Additional examples of bulky molecules include, for example, without limitation, biotin, azide groups and products of reactions with azide groups, one or more small molecules, one or more polyhistidine tags, and/or one or more proteins. Some embodiments further comprise introducing a bulky moiety into the splint oligonucleotide. Some embodiments further comprise introducing a bulky moiety on the template switching oligos; such bulky moieties may reduce concatemer formation. The bulky moiety may be introduced at a position that has the lowest effect on adapter attachment efficiency, such as the 5′-end region of the splint oligonucleotide, close to the 5′-end region of the splint oligonucleotide, or away from the ligation junction in ligation-based adapter attachment reactions.

As used herein, “control” refers to a standard of comparison. A “negative control” refers to a standard of comparison that is used to identify contaminants from samples or to identify the nature of a signal in the absence of a sample. A “positive control” refers to a standard of comparison that is used to identify normal substances from an initial sample or sample. Some embodiments of the disclosure comprise a positive and/or negative control. Some embodiments of the disclosure comprise an initial sample or samples without a positive and/or negative control. Some embodiments of the disclosure comprise an initial sample or samples without a positive control. Some embodiments of the disclosure comprise an initial sample or samples without a negative control.

As used herein, “denaturing” refers to a process in which biomolecules, such as proteins or nucleic acids, lose their native or higher order structure. Native and higher order structure may include, for example, without limitation, quaternary structure, tertiary structure, or secondary structure. For example, a double-stranded nucleic acid molecule can be denatured into two single-stranded molecules.

As used herein, the term “dephosphorylation” or “dephosphorylating” refers to removal of a terminal phosphate group, such as the 5′- and/or 3′-end phosphate, from a nucleic acid, such as DNA to generate 5′- and/or 3-hydroxyl groups.

As used herein, “detect” refers to quantitative or qualitative detection, including, without limitation, detection by identifying the presence, absence, quantity, frequency, concentration, sequence, form, structure, origin, or amount of an analyte.

As used herein, “digoxigenin” refers to a bulky molecule or its complex comprising the structure:

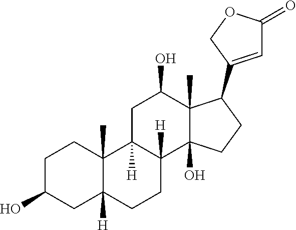

As used herein, “isolation” or “purification” and their cognates, of nucleic acids refers to steps (e.g., elution) after the start of and in the generation of a nucleic acid library that separate the nucleic acid from at least one component with which it is normally associated (e.g., a ligase or a polymerase).

As used herein, “removal” or “extraction,” and their cognates, of nucleic acids refers to steps prior to the start of generating or preparing a nucleic acid library that separate nucleic acids from at least one component with which they are normally associated. Removal or extraction of nucleic acids may refer to the process of creating an initial sample from a raw biological sample. For example, without limitation, the fractionation of whole blood into its component parts, such as plasma, may be considered to involve removal or extraction. Similarly, purification or isolation of DNA from a sample (e.g., plasma sample) can be considered extraction.

As used herein, “GC-bias” refers to differential performance (e.g., amplification) or treatment of nucleic acids having different GC content but identical length.

As used herein, “GC-content” or “guanine-cytosine content” refer to the percentage or quantity of nitrogenous bases in a nucleic acid, such as a DNA or RNA molecule, that are either guanine or cytosine or their chemical modifications.

As used herein, “host” refers to an organism that harbors another organism or microbe. For example, a living thing e.g. a mammal such as a human being can be a host that harbors a microbe or pathogen, the microbe or pathogen being the non-host.

As used herein, the phrase “identifying sequence element” or “identifying tag” refers to an element of a sequence that identifies an index, a code, a barcode, a random sequence, an adapter, an overhang of non-templated nucleic acids, a tag comprising one or more non-templated nucleotides, a priming sequence, unique molecular identifiers, or any combination thereof.

As used herein, “Klenow fragment” refers to a large protein fragment of DNA polymerase I that retains the 5′-----3′ polymerase activity and the 3′-----5′ exonuclease activity for removal of precoding nucleotides and proofreading but loses its 5′------3′ exonuclease activity.

As used herein, “ligating” or “ligation” refers to the joining of two ends of nucleic acid fragments through the action of an enzyme. DNA molecules and RNA molecules may be ligated. There are many methods of ligation and one skilled in the art would readily understand methods of ligation other than those disclosed herein.

As used herein, “length bias” refers to a bias with respect to length of a particular nucleic acid size or fragment length created by a sequencing library generation process as opposed to another size or fragment length. It may be preferable to reduce length bias for consistent or more accurate results. In some aspects, it may be preferable to increase a length bias for a certain range or against a certain range.

As used herein, “microbe,” or “microbial,” generally refers to bacteria, fungi, protists, parasites, viruses, or other entities that are usually detectable using a microscope. As used herein, the term “microorganism” refers to a uni- or multi-cellular organism, such as, for example, a microscopic organism or macroscopic organism including but not limited to bacteria, fungi, protists, and parasites. Microbes are often pathogens responsible for disease, but may also exist in a non-pathogenic, symbiotic, commensalistic, mutualistic, or amensalistic relationship with a host, such as a human.

Examples of microbes include one or more species or strains from one or more of the following genera: Coniosporium, Hantavirus, Talaromyces, Machlomovirus, Betatetravirus, Raoultella, Aeromonas, Ephemerovirus, Empedobacter, Loa, Macluravirus, Stenotrophomonas, Alfamovirus, Rosavirus, Emmonsia, Aggregatibacter, Orthopneumovirus, Weeksella, Nairovirus, Salivirus, Weissella, Mosavirus, Gammapartitivirus, Strongyloides, Passerivirus, Erysipelatoclostridium, Bacillamavirus, Iotatorquevirus, Taenia, Trypanosoma, Olsenella, Cladosporium, Rhizobium, Prevotella, Leclercia, Paracoccus, Ilarvirus, Lagovirus, Rasamsonia, Plasmodium, Acremonium, Chlamydia, Clonorchis, Vibrio, Bartonella, Nakazawaea, Franconibacter, Anisakis, Norovirus, Nocardia, Solobacterium, Parechovirus, Avenavirus, Orthohepevirus, Aphthovirus, Hepandensovirus, Microbacterium, Lichtheimia, Lomentospora, Achromobacter, Ipomovirus, Tsukamurella, Elizabethkingia, Hepevirus, Seadomavirus, Alternaria, Trueperella, Gammatorquevirus, Bifidobacterium, Chrysosporium, Thogotovirus, Curtovirus, Deltatorquevirus, Balamuthia, Mastrevirus, Bdellomicrovirus, Mupapillomavirus, Pseudozyma, Wickerhamiella, Aquamavirus, Alloscardovia, Thielavia, Idaeovirus, Henipavirus, Coxiella, Haemophilus, Gammacoronavirus, Negevirus Brevibacterium, Peptoniphilus, Alphacarmotetravirus, Nosema, Trichovirus, Arenavirus, Thermomyces, Necator, Waikavirus, Blosnavirus, Jonesia, Tetraparvovirus, Emaravirus, Plectrovirus, Sclerodamavirus, Toxocara, Umbravirus, Burkholderia, Chromobacterium, Paracoccidioides, Brugia, Eragrovirus, Macrococcus, Absidia, Colletotrichum, Inovirus, Phycomyces, Wickerhamomyces, Acidaminococcus, Moraxella, Rothia, Phlebovirus, Slackia, Purpureocillium, Betapapillomavirus, Tupavirus, Cryspovirus, Saksenaea, Erysipelothrix, Kobuvirus, Mimoreovirus, Echinococcus, Mannheimia, Bergeyella, Cyclospora, Xylanimonas, Leptospira, Finegoldia, Curvularia, Cryptosporidium, Babuvirus, Pecluvirus, Lambdatorquevirus, Pythium, Carlavirus, Entomobimavirus, Kocuria, Anaplasma, Ampelovirus, Avihepatovirus, Nepovirus, Rhodococcus, Bordetella, Mischivirus, Scedosporium, Gardnerella, Maculavirus, Trichoderma, Aveparvovirus, Salmonella, Avastrovirus, Copiparvovirus, Trachipleistophora, Clostridioides, Nanovirus, Siccibacter, Leptotrichia, Citrivirus, Odoribacter, Sanguibacter, Novirhabdovirus, Acremonium, Hafnia, Chaetomium, Tenuivirus, Yokenella, Rubulavirus, Varicellovirus, Alphamesonivirus, Sicinivirus, Leuconostoc, Microvirus, Gallantivirus, Morbillivirus, Lolavirus, Pantoea, Hepatovirus, Nupapillomavirus, Metschnikowia, Bamavirus, Kytococcus, Tritimovirus, Tannerella, Respirovirus, Pneumocystis, Dirofilaria, Pediococcus, Lactococcus, Blastomyces, Dianthovirus, Actinobacillus, Teschovirus, Oscivirus, Begomovirus, Potyvirus, Byssochlamys, Alphacoronavirus, Molluscipoxvirus, Lymphocryptovirus, Sapelovirus, Parabacteroides, Pyrenochaeta, Listeria, Senecavirus, Brevidensovirus, Potexvirus, Parvimonas, Flavivirus, Recovirus, Toxoplasma, Yatapoxvirus, Opisthorchis, Trichuris, Cyphellophora, Morganella, Perhabdovirus, Micrococcus, Pequenovirus, Mastadenovirus, Anaeroglobus, Tropheryma, Dolosigranulum, Wolbachia, Lelliottia, Mycoplasma Tobravirus, Shewanella, Paeniclostridium, Erythroparvovirus, Sutterella, Sporopachydermia, Namavirus, Nyavirus, Francisella, Arthroderma, Epsilontorquevirus, Sigmavirus, Amdoparvovirus, Actinomyces, Alphapermutotetravirus, Cardiobacterium, Influenzavirus C, Orthopoxvirus, Poacevirus, Phialophora, Lactobacillus, Polyomavirus, Debaryomyces, Foveavirus, Bymovirus, Mycoflexivirus, Grimontia, Mucor, Rhytidhysteron, Quadrivirus, Thermoascus, Aureusvirus, Trichosporon, Myceliophthora, Dermacoccus, Dysgonomonas, Pseudoramibacter, Becurtovirus, Gordonia, Sapovirus, Orthobunyavirus, Spiromicrovirus, Pomovirus, Exophiala, Sneathia, Helicobacter, Photorhabdus, Mogibacterium, Betapartitivirus, Avibirnavirus, Ambidensovirus, Oleavirus, Orientia, Deltacoronavirus, Anulavirus, Trichomonasvirus, Budvicia, Geotrichum, Enamovirus, Lachnoclostridium, Schistosoma, Paecilomyces, Panicovirus, Rhizoctonia, Brevibacillus, Beauveria, Pestivirus, Tombusvirus, Cilevirus, Cokeromyces, Peptostreptococcus, Phanerochaete, Proteus, Idnoreovirus, Aspergillus, Pasteurella, Malassezia, Hanseniaspora, Endomavirus, Azospirillum, Velarivirus, Cystovirus, Avisivirus, Bacteroides, Picobirnavirus, Myroides, Circovirus, Arterivirus, Aquaparamyxovirus, Onchocerca, Cosavirus, Kluyveromyces, Fijivirus, Candida, Hepacivirus, Dermabacter, Ourmiavirus, Allexivirus, Enterobacter, Acidovorax, Bracorhabdovirus, Carmovirus, Pluralibacter, Coltivirus, Fonsecaea, Streptobacillus, Corynebacterium, Macrophomina, Marburgvirus, Comovirus, Fabavirus, Alphanodavirus, Cellulomonas, Enterobius, Catabacter, Moellerella, Nakaseomyces, Cucumovirus, Valsa, Deltapartitivirus, Plesiomonas, Pseudomonas, Torovirus, Cuevavirus, Hypovirus, Trichomonas, Influenzavirus D, Giardiavirus, Crinivirus, Tepovirus, Sakobuvirus, Cyberlindnera, Paenalcaligenes, Bafinivirus, Rymovirus, Pegivirus, Yarrowia, Treponema, Borreliella, Rubivirus, Aureobasidium, Angiostrongylus, Filobasidium, Photobacterium, Rhizopus, Orthoreovirus, Ustilago, Simplexvirus, Aquareovirus, Protoparvovirus, Propionibacterium, Sprivivirus, Hunnivirus, Apophysomyces, Meyerozyma, Alphapapillomavirus, Candida, Brucella, Gallivirus, Dinovernavirus, Anaerobiospirillum, Eubacterium, Tatlockia, Terrisporobacter, Quaranjavirus, Sobemovirus, Dicipivirus, Arcanobacterium, Macanavirus, Atopobium, Vesivirus, Lodderomyces, Dinornavirus, Betatorquevirus, Kerstersia, Aparavirus, Neisseria, Agrobacterium, Edwardsiella, Labyrnavirus, Totivirus, Actinomadura, Tobamovirus, Influenzavirus B, Mandarivirus, Anaerococcus, Kunsagivirus, Naegleria, Campylobacter, Veillonella, Yamadazyma, Filobasidiella, Oerskovia, Penicillium, Anncaliia, Leptosphaeria, Pneumovirus, Psychrobacter, Isavirus, Granulicatella, Torradovirus, Cladophialophora, Influenzavirus A, Ophiostoma, Aerococcus, Ureaplasma, Etatorquevirus, Bocaparvovirus, Megasphaera, Reptarenavirus, Comamonas, Capnocytophaga, Alphatorquevirus, Syncephalastrum, Wallemia, Betacoronavirus, Hyphopichia, Nocardiopsis, Legionella, Trichinella, Paraburkholderia, Mammarenavirus, Echinostoma, Sphingobacterium, Enterovirus, Methanobrevibacter, Ochroconis, Cheravirus, Pasivirus, Enterococcus, Mycoreovirus, Tospovirus, Betanodavirus, Phytoreovirus, Enterocytozoon, Ferlavirus, StemphyliumFilifactor, Leishmaniavirus, Gemella, Bromovirus, Alloiococcus, Cunninghamella, Cronobacter, Oribacterium, Orbivirus, Chrysovirus, Cripavirus, Tatumella, Pandoraea, Ogataea, Dracunculus, Volvariella, flavirus, Benyvirus, Rhadinovirus, Histoplasma, Rahnella, Morococcus, Verticillium, Janibacter, Gyrovirus, Alphapartitivirus, Mycobacterium, Roseomonas, Varicosavirus, Chryseobacterium, Parapoxvirus, Rhizomucor, Aureimonas, Levivirus, Leishmania, Luteovirus, Cypovirus, Ochrobactrum, Microsporum, Piscihepevirus, Ceratocystis, Sporothrix, Vesiculovirus, Cupriavidus, Cryptococcus, Metapneumovirus, Alphanecrovirus, Eikenella, Brevundimonas, Escherichia, Leifsonia, Schizophyllum, Granulibacter, Gordonibacter, Lachancea, Madurella, Ophiovirus, Phellinus, Nebovirus, Acanthamoeba, Fusobacterium, Pichia, Verruconis, Ehrlichia, Tibrovirus, Higrevirus, Wohlfahrtiimonas, Rhinocladiella, Neorickettsia, Sadwavirus, Roseobacter, Sequivirus, Pannonibacter, Rotavirus, Turicella, Cardiovirus, Propionimicrobium, Furovirus, Naumovozyma, Closterovirus, Fluoribacter, Zeavirus, Clavispora, Megrivirus, Gammapapillomavirus, Rickettsia, Polemovirus, Corynespora, Encephalitozoon, Shimwellia, Fusarium, Yersinia, Capronia, Delftia, Victorivirus, Marafivirus, Kluyvera, Iteradensovirus, Isoptericola, Vitivirus, Roseolovirus, Conidiobolus, Abiotrophia, Babesia, Phoma, Sanguibacteroides, Staphylococcus, Rhodotorula, Zetatorquevirus, Hymenolepis, Fasciola, Cytorhabdovirus, Cardoreovirus, Memnoniella, Trichophyton, Mitovirus, Phaeoacremonium, Providencia, Lysinibacillus, Giardia, Oligella, Streptomyces, Paraclostridium, Ralstonia, Coccidioides, Brambyvirus, Biatriospora, Allolevivirus, Acinetobacter, Starmerella, Omegatetravirus, Porphyromonas, Avulavirus, Streptococcus, Arcobacter, Topocuvirus, Mamastrovirus, Ancylostoma, Bomavirus, Capillovirus, Alphavirus, Tymovirus, Nucleorhabdovirus, Diaporthe, Chlamydiamicrovirus, Tumcurtovirus, Saccharomyces, Riemerella, Betanecrovirus, Clostridium, Mobiluncus, Cercospora, Mamavirus, Mortierella, Aquabimavirus, Xanthomonas, Dependoparvovirus, Ebolavirus, Neofusicoccum, Borrelia, Leminorella, Klebsiella, Blastocystis, Alcaligenes, Citrobacter, Eggerthella, Cedecea, Serratia, Penstyldensovirus, Bacillus, Laribacter, Wuchereria, Hordeivirus, Cytomegalovirus, Actinomucor, Ascaris, Shigella, Vittaforma, Torulaspora, Kingella, Oryzavirus, Polerovirus, Tremovirus, Erbovirus, Entamoeba, Lyssavirus, Paenibacillus, Facklamia, Kappatorquevirus, Metarhizium, Stachybotrys, Okavirus, Botrexvirus, Thetatorquevirus, and Basidiobolus.

Microbes or pathogens may include archaea, bacteria, yeast, fungi, molds, protozoans, nematodes, eukaryotes, and/or viruses. Microbes or pathogens may also include DNA viruses, RNA viruses, culturable bacteria, additional fastidious and unculturable bacteria, mycobacteria, and eukaryotic pathogens (See, Bennett et al., Mandell, Douglas, and Bennett's Principles and Practice of Infectious Diseases, Ninth Edition; Elsevier, 2019; and Netter's Infectious Disease, 2nd Edition, Jong and Stevens, eds. Elsevier, 2021). Microbes or pathogens may also include any of the microbes known to a person of skill.

As used herein, “nucleic acid” refers to a polymer or oligomer of nucleotides and is generally synonymous with the term “polynucleotide” or “oligonucleotide.” Nucleic acids may comprise a deoxyribonucleotide, a ribonucleotide, a deoxyribonucleotide analog, chemically modified canonical deoxyribonucleotides, ribonucleotides, and/or ribonucleotide analog, nucleic acids with modified backbones, or any combination thereof.

Nucleic acids can be of any length. The following are non-limiting examples of nucleic acids: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), long non coding RNA (Inc RNA), small non coding RNAs such as but not restricted to piwi RNAs and enhancer RNAs, circ RNA (circular RNA), ribozymes, cDNA, recombinant polynucleotides, branched polynucleotides, plasmids, vectors, isolated DNA of any sequence, isolated RNA of any sequence, nucleic acid probes, primers, mitochondrial DNA, circulating nucleic acids, cell-free nucleic acids, cfDNA, cfna, CFNA, host cfNA, non-host cfNA, circulating cfNA, microbial cell free nucleic acids, viral nucleic acid, bacterial nucleic acid, genomic DNA, pathogen nucleic acids, fungal nucleic acid, parasitic nucleic acid, exosomal nucleic acid, intercellular signal nucleic acid, exogenous nucleic acids, nucleic acid therapeutics, and DNA enzymes. A nucleic acid may comprise one or more modified nucleotides, such as methylated nucleotides or methylated nucleotide analogs. If present, modifications to the structure may be imparted before or after assembly of the polymer. The sequence of nucleotides may be interrupted by non-nucleotide components. A nucleic acid may be further modified after polymerization, such as by conjugation with a labeling component. A nucleic acid may be single-stranded, double-stranded, have higher numbers of strands (e.g., triple-stranded), and/or have a higher order of structure (e.g., tertiary, or quaternary structure). A target nucleic acid may be any type, category, or subcategory of nucleic acids.

As used herein, a “nucleic acid library” refers to a collection of nucleic acid fragments. The collection of nucleic acid fragments may be used, for example, for sequencing.

As used herein, “pathogen” refers to a microbe that can cause a disease, ailment, or an infection.

As used herein, “plasma” or “blood plasma” refers to the liquid component or fraction of blood. Plasma is generally obtained by spinning a whole blood sample and removing the liquid component.

As used herein, the phrase “process control molecules” refers to molecules that are added to a sample before or during nucleic acid library generation to aid in the identification or quantification of nucleic acids in a sample. Process control molecules are separate from and not integrated in the target molecules, such as nucleic acids. Process control molecules may have special features such as specific sequences, lengths, GC content, degrees of degeneracy, degrees of diversity, different secondary, tertiary, or quaternary structures, and/or known starting concentrations. Process control molecules may be used for normalizing the signal in a sample in order to account for variations in sample processing or to control process performance. Process control molecules may include, for example, without limitation, ID Spike(s), Spanks, and/or Sparks or GC Spike-in Panel molecules. Process control molecules may additionally include dephosphorylation control molecules, denaturation control molecules, and/or ligation control molecules. Examples of dephosphorylation control molecules include, without limitation: GGCCTCGCGGAGGCATGCGTCATGCTAGCGTGCGGGGTACTCTTGCTATC (SEQ ID NO: 4); GAGAATTATTCGGGGGCAGTGACAACCAACATCTCGGGTCCTGCCCAACC-3′Phosph (SEQ ID NO: 5); 5′Phosph-GGTCTACACGCTAATATAGCGAATCACCGAGAACCCGGCGCCACGCAATG-3′Phosph (SEQ ID NO: 6); and 5′Phosph-GAACGTCCTTAACTCCGGCAGGCAATTAAAGGGAACGTATGTATAACGCA (SEQ ID NO: 7), where “5′Phosph” and “3′Phosph” indicate that the 5′-end and 3′-end of the control molecule is phosphorylated, respectively. By “adapter attachment control molecule” is intended a control molecule that allows monitoring of the efficiency of an adapter attachment reaction be it ligation-based, TdT-based, template-switching-based, primer-extension-based, or amplification-based. By “degradation assessment molecules” is intended a control molecule used to evaluate sample and spiked sample integrity during processing.

The term “sequencing,” as used herein, generally refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA). Sequencing may involve basic methods including Maxam-Gilbert sequencing and chain-termination methods, or de nova sequencing methods including shotgun sequencing and bridge PCR, or next-generation sequencing (NGS) methods (or massively-parallel sequencing method) including but not limited to polony sequencing, 454 pyrosequencing, Illumina sequencing, SOLiD sequencing, Ion Torrent semiconductor sequencing, HeliScope single molecule sequencing, SMRT® sequencing, nanopore sequencing and others. Sequencing can be performed by various systems currently available, such as, without limitation, a sequencing system by Illumina, Pacific Biosciences, Oxford Nanopore, Genia Technologies, or Life Technologies (Ion Torrent) and others. Such devices may provide a plurality of raw genetic data corresponding to the genetic information of a host (e.g., human), a non-host (e.g., a pathogen, an organ donor), a host-derived variant genetic sequence (e.g., a single nucleotide polymorphism), and/or combinations thereof as generated by the device from a sample provided by the subject

As used herein, “Spanks” refers to degenerate pools, or pools of nucleic acids with diverse sequences, which degenerate pools may often be used for diversity assessment, abundance calculation, and/or determination of information transfer efficiency (See, for example, U.S. Pat. No. 9,976,181).

As used herein, “Sparks” “GC Spike-in Panel” or “GC dSPARKS” refers to nucleic acids that are size or length or GC-content markers, which may be used for abundance normalization, development, and/or analysis purposes and other purposes (See, for example, U.S. Pat. No. 9,976,181).

As used herein, “ID Spike(s)” refers to identification spikes that can be used, for example without limitation, for sample identification tracking, cross-contamination detection, reagent tracking, and/or reagent lot tracking (See, for example, U.S. Pat. No. 9,976,181).

As used herein, the phrase “raw biological sample” refers to an unmanipulated sample obtained from a subject, e.g., host, containing or presumed to contain target nucleic acids. In other words, a raw biological sample, once obtained from the subject, has not been subjected to any extraction methods, e.g., alcohol-based extraction, size separation, etc., needed to generate an initial sample. Exemplary raw biological samples include whole blood, cerebrospinal fluid, synovial fluid, bronchoalveolar lavage, urine, stool, saliva, abdominal fluid, ascites fluid, peritoneal lavage, gastric fluid, interstitial fluid, lymph fluid, bile, abscess fluid, tissue, amniotic fluid, meconium, sinus aspirate, lymph node, bone marrow, hair, nails, cheek swab, skin swab, urethral swab, cervical swab, nasopharyngeal swab, nasopharyngeal aspirate, vaginal swab, epithelial cells, semen, vaginal discharge, intercellular fluid, pericardial fluid, rectal swab, bone, skin tissue, soft tissue, tears, and/or a nasal sample. The raw biological sample may be an initial sample if no manipulation of the raw biological sample is needed, e.g., whole blood, to obtain the target nucleic acids. A raw biological sample may also be manipulated, such as, for example to create a fraction of whole blood (e.g., plasma, serum, etc.) to yield an initial sample.

As used herein, the term “initial sample” refers to a sample comprising nucleic acids derived from a raw biological sample. An initial sample, for example, may comprise target or desired nucleic acids obtained or extracted from a raw biological sample.

As used herein, the phrase “spiked initial sample” refers to an initial sample to which process control molecules have been added prior to the start of generating a sequencing library.

The term “derived from” encompasses the terms “originated from,” “obtained from,” “obtainable from” and “created from,” and generally indicates that one specified material finds its origin in another specified material or has features that can be described with reference to the specified material. For example, an initial sample may be derived from a raw biological sample.

As used herein, the turn of phrase “uniformly distributed” refers to a distribution that is continuous or uniform between members of a family such that for each member of a family there is a predictable or symmetric interval between them. The term “non-uniformly distributed” refers to a distribution of members of a family that does not have a predictable or symmetric interval between them.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited herein. Additional embodiments are disclosed in further detail in the following examples, which are provided by way of illustration and are not in any way intended to limit the scope of this disclosure or the claims.

### Example 1: Preparation of a Nucleic Acid Library from a Biological Fluid of a Subject by Exploiting Differential Amenability to Ligation of Two Populations of Nucleic Acids

This disclosure generally shows that human cell-free nucleic acids tend to contain a higher degree of 5′ phosphorylation than microbial cell-free nucleic acids derived from certain microbes. Many of the methods disclosed herein exploit this difference in order to enrich for non-human nucleic acids in a sample, particularly microbial cell-free nucleic acids.

In the present example, a plasma sample is collected from a human subject and spiked with process control molecules. The plasma sample contains human cfDNA, which is generally natively phosphorylated at the 5′ end, and therefore amenable to ligation at the 5′ end. Also present in the plasma sample is microbial cell-free DNA (mcfDNA) derived from certain microbes; the mcfDNA from certain microbes generally contains less native 5′ phosphorylation and is therefore less susceptible to 5′ ligation. The cell-free DNA is obtained or extracted from the plasma yielding a sample comprising a mixture of obtained or extracted double-stranded and single-stranded DNA. The DNA is treated with proteinase K enzyme to remove histones and other proteins and heated (95° C.) to yield a sample generally containing single-stranded DNA (FIG. 1A).

In order to attach an oligonucleotide to the 3′ end of the cell-free DNA, a 3′ splint oligonucleotide (“3′ splint oligo” or 3′ adapter) 101 is added to the sample; and a ligation reaction is performed in order to ligate the 3′ splint oligo 101 to the 3′ end of the single-stranded DNA in the sample. As shown in FIG. 1B, the 3′ splint oligo 101 contains (a) a double-stranded region (102, 103) that comprises a 3′ ligation oligo 102 and a 3′ ligation oligo complement 103) and (b) a single-stranded region 104 (or overhang region) containing a random sequence of nucleotides (“random region”), wherein the single-stranded region 104 is connected to the 3′ ligation oligo complement 103. Stated a different way, the 3′ splint oligo 101 contains a “splint” region 105 that contains a random region 104, that is connected, at its 5′ end, to the 3′ ligation oligo complement 103. The random regions in a collection of 3′ splint oligo's can enable the splint oligo's to bind to a large number of different target nucleic acids in a sample (e.g., genomic cell-free nucleic acids). In FIG. 1B, the 3′ ligation oligo 102 is phosphorylated at its 5′ end to permit ligation to the 3′ end of the cell-free DNA. Another feature of the splint oligo's is that the splint region, 105, generally can comprise one or more uracil bases that are susceptible to digestion by USER enzyme. As further described herein, the splint region (114) of the 5′ splint dummy oligo and the splint region (124) of the 5′ splint oligo, can also have one or more uracil bases that are susceptible to digestion by USER enzyme.

As shown in FIG. 1A, a 5′ ligation reaction is performed to selectively modify the 5′ phosphorylated ends of the human cell-free DNA with a blocking oligonucleotide (e.g., “dummy oligo”). In some cases, an oligo with an identifiable sequence is used instead of a dummy oligo, along with a dummy oligo or as part of the dummy oligo. In some cases, the oligo with the identifiable sequence can be used to mark or identify a particular sequence, for example, to mark a sequence as “human.” The 5′ ligation can occur before, concurrently, or following the 3′ ligation of the 3′ splint oligo. As shown in FIG. 1B, the 5′ dummy oligo 110 generally has the same structure as the 3′ splint oligo 101 in that it has a double-stranded region (111-112) and a single-stranded region (or overhang region) containing a random sequence of nucleotides 113. In the splint region of the 5′ dummy oligo 114 the random sequence 113 is connected at its 3′ end to the 5′ dummy oligo complement 112. In order to prevent subsequent ligation, the 5′ dummy oligo (or 5′ dummy ligation oligo) is not phosphorylated at its 5′ end (depicted by a circle 115) and is also resistant to phosphorylation, e.g., by a polynucleotide kinase (PNK).

Next, T4 polynucleotide kinase (PNK) is introduced to the sample in order to phosphorylate the 5′ ends of the cell-free nucleic acids, except for the cell-free DNA blocked the 5′ dummy oligo. As a result, microbial cell-free DNA is preferentially phosphorylated at the 5′ end, while the dummy oligo selectively prevents the human cell-free DNA from becoming phosphorylated at its 5′ end. A 5′ splint oligo 120 is then added to the sample. The 5′ splint oligo 120 generally has the same structure as the 5′ dummy splint oligo 110 except that its 5′ end does not need to be resistant to phosphorylation. As shown in FIG. 1B, the 5′ splint oligo 120 has a double stranded region (121-122) and a single-stranded region (or overhang region) containing a random sequence of nucleotides 123. The splint region (124) contains a random sequence of nucleotides 123 (or random region) connected at its 3′ end to the 5′ ligation oligo complement 122.

The 5′ splint oligo is then subjected to a ligation reaction that preferentially ligates the 5′ splint oligo to cell-free DNA that is not attached to the dummy oligo (in other words, cfDNA that is preferentially microbial cfDNA and that was subjected to phosphorylation by PNK). The biological sample is subjected to a USER digestion reaction to cleave the uracil bases in the splint oligo's in order to yield single-stranded DNA that is attached to 5′ and 3′ single-stranded ligation oligos (or adapters). Next, the single stranded DNA that is attached to the single strand ligation oligos in the sample are PCR amplified using primers that recognize the 5′ ligation oligo (121) and the 3′ ligation oligos (102) (or 5′ and 3′ adapters). In some cases, additional adapters are attached to the 5′ ligation oligo and/or the 3′ ligation oligo and the PCR amplification is conducted using primers that recognize the additional adapters. The result is a library preferentially enriched for mcfDNA and fully amenable to downstream processing including sequencing and sequence analysis as disclosed herein.

### Example 2: Materials and Methods

### A. Spike-In Master Mix:

A set of process control molecules are pre-mixed together in a single Spike-in Master Mix, with each Spike-in Master Mix containing a unique “ID Spike” process control molecule. Spike-in Master Mix contain three classes of molecules; ID Spike molecules, SPANK molecules, and SPARK molecules. The latter group of molecules are composed of two classes of SPARKs: GC dSPARKs and Long SPARKs. The molar concentration of ID Spike, SPANK molecules, and long SPARK molecules in the Spike-in Master Mix is 10 picomolar (pM) per molecule. The molar concentration of GC dSPARK molecules is about 1 pM per molecule.

### B. “ID Spike” Molecules

Each sample is labeled with a unique ID Spike double-stranded DNA molecule that is characterized by a 100 base pairs long unique sequence that was not present in any reference genome available in a public database at the time of processing.

### C. SPANK Molecules

A pool of double-stranded DNA molecules (“SPANK” molecules) is added to the sample, each SPANK molecule is 75 base pairs long with identical 3′-end and 5′-end sequences that were not present in any reference genome available in public databases at the time of processing. In addition, two stretches of 8 base pairs nested between the constant 3′-end and 5′ end sequences were present and fully degenerate within the pool. The pool of SPANK molecules contained 4¹⁶ unique SPANK molecules. The two degenerate stretches were separated by a stretch of four non-degenerate bases.

### D. SPARK Molecules

A GC Spike-in Panel can also be added to the sample. The GC spike=in panel is a set of molecules that are 32, 42, 52, and 75 base pairs long where 7 different sequences with GC content 20%, 30%, 40%, 50%, 60%, 70%, and 80% are included for each length. Like some of the other molecules provided above, GC dSPARK sequences do not occur in the available reference genomes. A Long SPARK sequence set is a group of 4 non-natural sequences, each with 50% GC content and lengths of 100 base pairs, 125 base pairs, 150 base pairs, and 175 base pairs. A complete set of SPARK molecules contained 32 different sequences.

### E. Sequencing Library Generation

Sequencing libraries are prepared following the process outlined below. For the direct-to-library process, 5 μL of spiked asymptomatic plasma is used as the library input.
Step 2.1: Manufacturing 3′-end adapter hybrid. Oligomers A and B (Table 1) purchased from IDT (Coralville, Iowa) are dissolved individually in 1×IDTE (IDT, Coralville, Iowa) at final concentration of 100 μM. Before use, the oligomers are enzymatically purified in separate reactions by mixing 200 μL of 100 μM oligomer A or B, 100 μL 10×NEBuffer 2 (NEB, Ipswich, Mass.), 10 μL Klenow fragment (50 u/μL, NEB, Ipswich, Mass.), 50 μL T4 PNK (10 u/μL, Thermo-Fisher Scientific, Waltham, Mass.), and 640 μL Nuclease-free water, followed by incubation at 37° C. for 20 min, and thermal deactivation at 95° C. for 1 min. After thermal deactivation, the enzymatically purified oligos A and B are mixed in an equimolar ratio without prior purification, and hybridized under the following thermal program: 95° C. for 10 s, cooling to 14° C. at 0.1° C./s, 5 min 14° C., cool down to 4° C. The 3′-end adapter hybrid is stored at −20° C. before use.
Step 2.2: Manufacturing 5′-end adapters. Oligomers C and D (Table 1) purchased from IDT (Coralville, Iowa) are dissolved individually in 1×IDTE (IDT, Coralville, Iowa) at final concentration of 500 μM. In order to obtain 5′-end adapter hybrid, 200 μL of 500 μM oligomer C and 200 μL of 500 μM oligomer D are mixed together and supplemented with 80 μL of Hybridization Buffer (0.95× TE, 250 mM NaCl). The oligomer annealing is conducted under the following temperature reaction: 95° C. for 10 s, cooling to 14° C. at 0.1° C./s, 5 min 14° C., cool down to 4° C. The resulting 5′-end adapter hybrid solution is then diluted with equal volume of 1×IDTE and stored at −20° C. before use. 5′-end adapters may be attached to a 3′-end of a complementary nucleic acid sequence.
Step 2.3: Dephosphorylation and denaturation. 5 μL of spiked plasma sample is mixed with 41.5 μL of dephosphorylation master mix (8.0 μL 10× T4 RNA ligase reaction buffer (NEB, Ipswich, Mass.), 0.2 μL 10% TWEEN®20, 1.0 μL FastAP (1 u/μL, Thermo-Fisher Scientific, Waltham, Mass.), 31.4 μL nuclease-free water), mixed well, and incubated at 37° C. for 10 min, followed by 2 min incubation at 95° C. to denature the molecules in the samples. While still at 95° C. the reaction mixture is transferred immediately and placed on ice to prevent renaturation of the nucleic acids. The library reactions using unprocessed spiked asymptomatic plasma as their inputs are centrifuged at 1600 g's, and 4° C. for 5 min to pellet the precipitate. Supernatant is collected and used in the next reaction step.
Step 2.4: 3′-end adapter ligation. 32 μL 50% PEG-8000, 0.4 μL 100 mM ATP, 1 μL 10 μM 3′-end adapter hybrid, and 1 μL T4 DNA Ligase (30 u/μL, Thermo-Fisher Scientific, Waltham, Mass.) are added to the reaction mixture resulting from the previous step. The mixture is mixed well, and incubated at 37° C. for 30 min, followed by denaturation and inactivation step at 95° C. for 1 min.
Step 2.5: Magnetic bead immobilization. Per library reaction, 20 μL of Dynabeads™ MyOne™ Streptavidin Cl magnetic beads are pre-washed with bead-binding buffer (1 M NaCl, 10 mM Tris-HCl (pH 8.0), 1 mM EDTA (pH 8.0), 0.05% TWEEN®20 and 0.5% SDS) and resuspended in 250 μL of bead-binding buffer. The resuspended magnetic beads are added to the final reaction mix from Step 2.2. The resulting mixture is incubated at ambient temperature for 20 min while constantly keeping the beads homogeneously dispersed in the mix. After a 20 min incubation, the tube with the sample is placed on a magnet stand to pellet the beads. The supernatant is collected and discarded. The remaining magnetic beads are washed with 200 μL Buffer A (0.1 M NaCl, 10 mM Tris-HCl (pH 8.0), 1 mM EDTA (pH 8.0), 0.05% TWEEN®20 and 0.5% SDS), the magnetic beads are pelleted, and the supernatant discarded. The remaining magnetic beads are resuspended in 100 μL stringency buffer (0.1×SSC and 0.1% SDS), and the suspension is incubated at 45° C. for 4 min, when it is placed on the magnet stand to pellet the beads. The supernatant is discarded, the remaining beads are resuspended in 200 μL Buffer B (0.1 M NaCl, 10 mM Tris-HCl (pH 8.0), 1 mM EDTA (pH 8.0) and 0.05% TWEEN®20) and pelleted on the stand. The supernatant is discarded, and the remaining beads are resuspended in the primer extension master mix (see Step 2.4).
Step 2.6: Primer Extension. The pelleted magnetic beads are resuspended in primer extension master mix (5 μL 10×10× REact® 2 Buffer (Thermo-Fisher Scientific, Waltham, Mass.), 0.4 μL 25 mM per each dNTP mix (Thermo-Fisher Scientific, Waltham, Mass.), 0.25 μL 10% TWEEN®20 and 1 μL 100 μM extension primer (Table 1), 41.35 μL nuclease-free water). The resulting suspension is incubated at 65° C. for 2 min, and immediately placed on ice. 2 μL klenow fragment (4 u/μL, Thermo-Fisher Scientific, Waltham, Mass.) is added to the mixture, mixed well, and placed at 25° C. for 5 min, while keeping the suspension homogeneous. After 5 min, the temperature is raised to 35° C., and incubated for 25 min, while keeping the suspension homogeneous. After, the suspension is placed on the magnet stand to pellet the beads. The supernatant is collected and discarded. The remaining magnetic beads are washed with Buffer A (0.1 M NaCl, 10 mM Tris-HCl (pH 8.0), 1 mM EDTA (pH 8.0), 0.05% TWEEN®20 and 0.5% SDS), the magnetic beads are pelleted, and the supernatant is discarded. The remaining magnetic beads are resuspended in Stringency Buffer (0.1×SSC and 0.1% SDS), and the suspension is incubated at 45° C. for 4 min, when it is placed on the magnet to pellet the beads. The supernatant is discarded, the remaining magnetic beads are resuspended in Buffer B (0.1 M NaCl, 10 mM Tris-HCl (pH 8.0), 1 mM EDTA (pH 8.0) and 0.05% TWEEN®20) and pelleted on a magnet. The supernatant is discarded, and the remaining magnetic beads are resuspended in the 5′-end ligation master mix (see Step 2.5).
Step 2.7: 5′-end adapter-ligation. The pelleted beads are resuspended in 5′-end ligation master mix (10 μL 10× T4 DNA Ligase Buffer (Thermo-Fisher Scientific, Waltham, Mass.), 10 μL 50% PEG-4000 (Thermo-Fisher Scientific, Waltham, Mass.), 2 μL 100 μM 5′-end adapter hybrid, 0.25 10% TWEEN®20, 0.332 μL T4 DNA ligase (30 u/μL, Thermo-Fisher Scientific, Waltham, Mass.), 75.9 μL nuclease-free water), and incubated at 25° C., while keeping the suspension homogeneous. After incubation, the suspension is placed on a magnet stand to pellet the beads. The supernatant is collected and discarded. The remaining beads are washed with Buffer A (0.1 M NaCl, 10 mM Tris-HCl (pH 8.0), 1 mM EDTA (pH 8.0), 0.05% TWEEN®20 and 0.5% SDS), pelleted, and the supernatant is discarded. The remaining magnetic beads are resuspended in Stringency Buffer (0.1×SSC and 0.1% SDS) and the suspension is incubated at 45° C. for 4 min, then placed on the magnet to pellet the beads. The supernatant is discarded, the remaining magnetic beads are resuspended in Buffer B (0.1 M NaCl, 10 mM Tris-HCl (pH 8.0), 1 mM EDTA (pH 8.0) and 0.05% TWEEN®20) and pelleted on a magnet. The supernatant is discarded, and the remaining magnetic beads are resuspended in the library elution buffer (see Step 2.8).
Step 2.8: Library elution. The pelleted magnetic beads are resuspended in 50 μL library elution buffer (10 mM Tris-HCl (pH 8.0), 0.1 mM EDTA, and 0.05% TWEEN®20), and incubated at 95° C. for 1 min to elute the library fragments off the beads. Next, the solution is placed immediately on a magnet to pellet the beads. 48 μL of the supernatant is then collected and placed in a new tube for amplification (see Step 2.9).
Step 2.9: Library Amplification. The library fragments in the eluate are amplified using ACCUPRIME™ Pfx DNA Polymerase (Thermo-Fisher Scientific, Waltham, Mass.) according to the manufacturer's recommendations, supplemented with indexing P5 and P7 primers purchased from IDT (Coralville, Iowa). Then the products are amplified using a thermocycler with the following temperature cycling program: Initial denaturation at 95° C. for 2 min, followed by 12 PCR cycles composed of cycle denaturation at 95° C. for 15 sec, cycle primer annealing at primer annealing at 60° C. for 30 sec, and primer extension at 68° C. for 1 min. In the final cycle, an extended primer extension step of 2 min is applied before cooling down to 4° C. Finally, the samples are purified using AGENCOURT® Rnaclean® XP magnetic beads per the manufacturer's recommendations and quantified using HS D1000 ScreenTape System (Agilent Technologies, Santa Clara, Calif) before running on a sequencer.

### F. Sequencing and Attribution of Reads

The samples are sequenced to obtain sequence reads using a NEXTSEQ™ 500 sequencer by Illumina. Sequencing is conducted following the manufacturer's instructions using Sequencing Primer (Table 1) as a custom Read 1 primer.

Primary sequencing output is demultiplexed by bcl2fastq v2.17.1.14 (with default parameters). Reads are aligned against human and synthetic (including process control molecules and sequencing adapter) references using Bowtie v2.2.4. Reads with alignments to either are set aside. Reads potentially representing human satellite DNA are also filtered via a k-mer based method. The remaining reads are aligned against a microorganism reference database using BLAST v2.2.30. Reads with alignments that exhibited both high percent identity and high query coverage are retained, with the exception of reads that align against any mitochondrial or plasmid reference sequences. PCR duplicates are removed based on their alignments.

Relative abundances are assigned to each taxon in a sample based on the sequencing reads and their alignments. For each combination of read and taxon, a read-sequence probability is defined that accounted for the divergence between the microorganism present in the sample and reference assemblies in the database. A mixture model is used to assign a likelihood to the complete collection of sequencing reads that include the read sequence probabilities and the (unknown) abundances of each taxon in the sample. An expectation-maximization algorithm is applied to compute the maximum likelihood estimate of each taxon abundance. From these abundances, the number of reads arising from each taxon are aggregated up the taxonomic tree.

A set of libraries is prepared from the respective negative control buffers and processed and sequenced within each batch. Estimated taxon abundances from the negative control samples within the batch are combined to parameterize a model of read abundance arising from the environment with variations driven by counting noise. Statistical significance values are computed for each estimated taxon abundance and those within the CRR at high significance levels comprised candidate calls (i.e., significant calls). Final calls (i.e., reportable calls) are made after additional filtering is applied, accounting for read location uniformity, read percent identity, and cross-reactivity originating from higher abundance calls.

Quantification of electrophoretic signal may reveal a mean library yield of direct-to-library process. Nucleic acids shorter than 100 base pairs may be represented, on average 69%±2% of the libraries obtained with the direct-to-library process.

### Example 3: Double Ligation Single Stranded DNA Library

What is disclosed herein is an alternative ssDNA library prep method with many of the same characteristics of Examples 1 and 2 to determine if short fragments are recovered or they are size selected out with the primer dimer peaks.

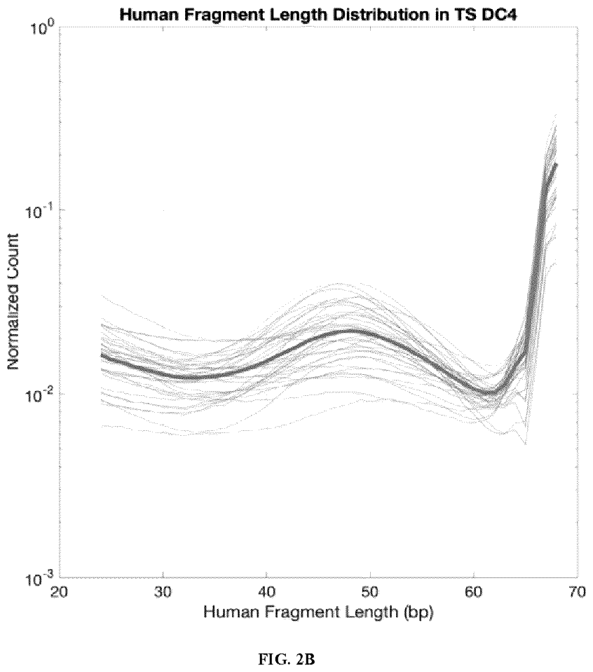

Two sets of duplexed oligos, one with a randomer, followed by Illumina sequence and the other the reverse complement of the Illumina sequence are added along with PNK, T4 ligase, ATP and PEG8000. These oligo duplexes are then ligated to both the 3′ and 5′ end of the cfDNA fragment. The 3′ and 5′ most bases of the oligos that are ligated to the cfDNA have had their bonds replaced with phosphorothioate bonds, rendering them resistant to exonucleases. Properly ligated cfDNA molecules will have these resistant oligos on both ends while all other products will not be fully protected. RecJf and Exo I are then added, digesting all of the unprotected species. Finally, standard indexing PCR is performed, followed by Ampure purification and sequencing. The results that may be obtained are shown in FIG. 2A-2B. Both single stranded library preparation methods show the 50 bp peak, in addition to the nucleosome.

The results of FIG. 2B show that the 50 bp peak is at least partly single-stranded cfDNA. Further, since the cfssDNA is predominantly human-derived, sensitivity may not increase using a method that relies on keeping the strands together.

Fragment length distribution is measured for pathogen cfDNA. The results show pathogen fragment length is more variable than human cfDNA. Most pathogen cfDNA has a 50 bp peak component. Environmental contamination (EC) pathogens are often short, exponentially distributed.

### Example 4: Differential Phosphorylation in Human Versus Microbial Cell-Free DNA

The vast majority of cfDNA found in human plasma is human of origin and further methods are needed to reduce this human signal in order to identify microbial cfDNA. One method to achieve this goal is to find a molecular difference between pathogen and human cfDNA.

In the present example, the methods disclosed in Examples 1 and 2 were used to detect cell-free DNA in a plasma sample (except without using a dummy oligonucleotide). Here, a human plasma sample was spiked with pathogen DNA, yielding a sample that contained human cell-free DNA, native microbial cell-free DNA (endogenous), and spiked pathogen DNA (exogenous). As noted in the previous examples, the method generally involves ligating 3′ splint oligonucleotides to the 3′ terminus of cell-free DNA obtained or extracted from the sample. The 3′ splint oligo's contain a 5′ phosphate group to facilitate a ligation reaction, which is mediated by T4 DNA ligase.

In order to ligate a 5′ splint oligonucleotide (or adapter) to the 5′ end of the cfDNA, T4 polynucleotide kinase (PNK) is used to phosphorylate the 5′ end of cell-free DNA thereby providing an appropriate substrate for T4 DNA ligase. If PNK is omitted from this reaction, then only DNA fragments that are natively phosphorylated are properly ligated to adapters and converted into a sequence-able form.

In this experiment, when PNK was omitted, the spiked pathogen DNA (known to have 5′ P) and the nucleosomal DNA (5′ P status unknown) were enriched in the library, while Sparks (no 5′ P) and endogenous pathogen (5′ P status unknown) were greatly depleted.

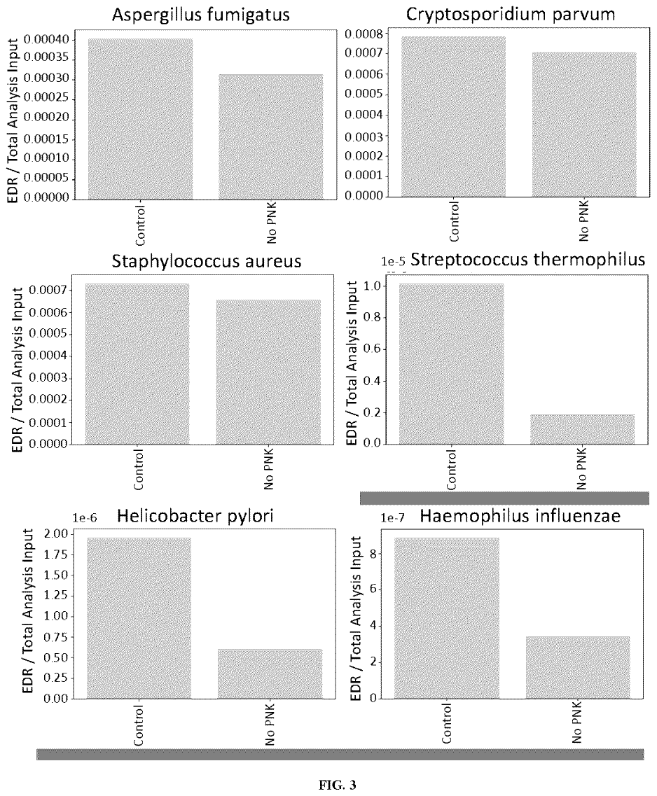

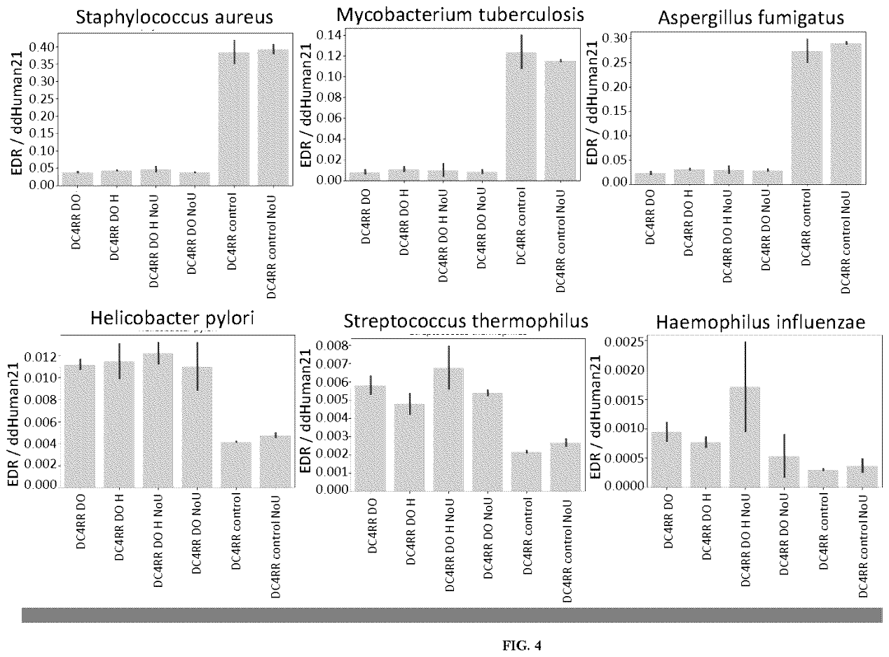

FIG. 3 shows the differential phosphorylation of human cell-free DNA (and spiked pathogen DNA) versus native, microbial cell-free DNA. Plasma from healthy human subjects was spiked with pathogen DNA. The spiked pathogens included Aspergillus funigatus, Cryptosporidium parvum, and Staphylococcus aureus. Endogenous microbes are shown by the thick horizontal bar and include Streptococcus thermophilus, Heliobacter pylori, and Haemophilus influenza. Duplicate reads, assumed to be derived from PCR duplication or sequencing instrument error, are identified based on alignment, and removed in a process we refer to as deduping. As a result of this process, the count of estimated unique or deduped reads is obtained by mapping to a particular pathogen reference. The relative abundance of microbes is expressed as estimated deduped reads (EDR), or reads per million (RPM, normalized to total reads for the sample), or reads per volume of sample (MPM, microbes per microliter). The ratio of microbial cfDNA (estimated deduped reads, EDR) to total human reads (EDR/ddHuman) is given without (no PNK, right) and with phosphorylation by PNK (control, left).

The results show a three- to five-fold depletion of native microbial cell-free DNA in the absence of PNK phosphorylation, indicating that native microbial cell-free DNA tends to have less 5′ phosphorylation than human cell-free DNA. Such a differential was less apparent for the microbial DNA that was spiked into the sample, indicating that the spiked DNA had levels of phosphorylation that was more comparable to that of human cell-free DNA.

These molecular differences can be used to preferentially enrich or deplete specific fractions of cfDNA. One method uses lambda exonuclease, which requires a 5′ P for its exonuclease activity and should preferentially degrade nucleosomal and spiked pathogen DNA. Another such method is described in a different Example herein and involves ligation of a 5′ “dummy” splint oligo to the 5′ termini of fragments that have a phosphate group, effectively blocking those fragments from the action of the ligase. PNK is then used to phosphorylate the remaining nucleic acids, which are preferentially microbial cell-free DNA. A second 5′ splint oligo, which is amplifiable, is then used to amplify and sequence the mcfDNA.

### Example 5: Enrichment of mcfDNA from Healthy Human Plasma by Using Dummy Oligonucleotides to Reduce Addition of Adapters to Cell-Free Human DNA

FIG. 4 shows detection of microbial nucleic acids when 5′ dummy splint oligonucleotides were used during sample processing of cell-free DNA from healthy human plasma. In this example, the methods of Example 1 and 2 were substantially used. Here, the 5′ dummy oligo was added to the 3′ ligation master mix. During the ligation reaction, the dummy 5′-oligo reacts with 5′-phosphorylated DNA at the same time as the 3′-ligation oligo or in a separate step. As a result, any cfDNA that is natively 5′-phosphorylated (including some human cfDNA and spiked pathogen DNA) is attached to a non-amplifiable 5′ dummy oligo. Following treatment with PNK, a second 5′-splint oligo was preferentially ligated to the 5′-termini of mcfDNA. Primers were added that recognize a nucleic acid sequence (or adapter sequence) present in the 5′-splint oligo. The microbial cell-free DNA was then preferentially amplified by PCR.

In each graph, the four left-most data values reflect EDR/ddHuman after using dummy oligonucleotides during sample processing, as described herein. Spiked pathogens including Staphylococcus aureus, Mycobacterium tuberculosis, and Aspergillus fumigatus (upper panels), were detected with a large reduction in sensitivity when dummy oligo's were used (FIG. 4 upper panels, left four vs. right two). In contrast, endogenous pathogens such as Heliobacter pylori, Streptococcus thermphilus, and Haemophilus influenzae (lower panel), were detected with a three-fold increase in sensitivity by EDR/ddHuman (FIG. 4 lower panels, left four vs. right two).

### Example 6: Enrichment of mcfDNA from Plasma from Infected Patients by Using Dummy Oligonucleotides to Reduce Addition of Adapters to Cell-Free Human DNA

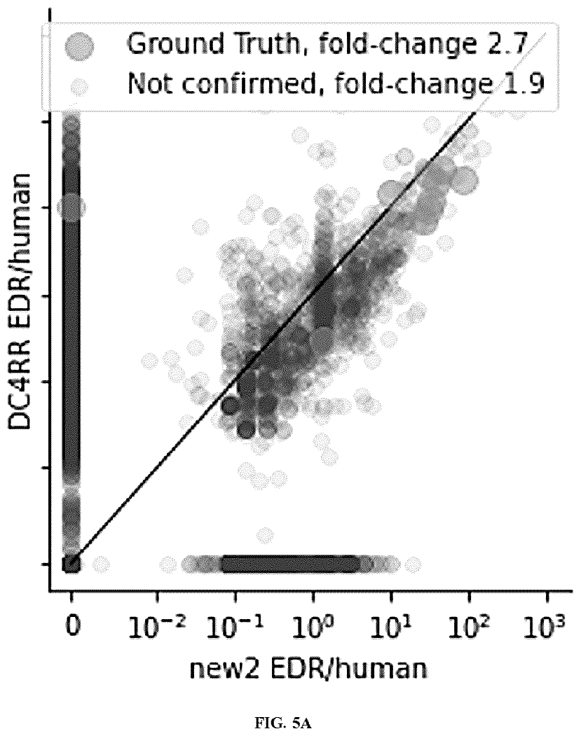

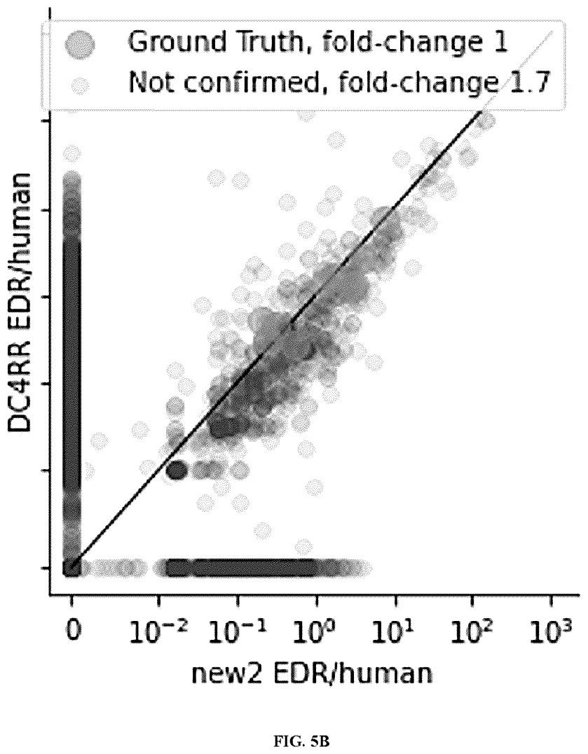

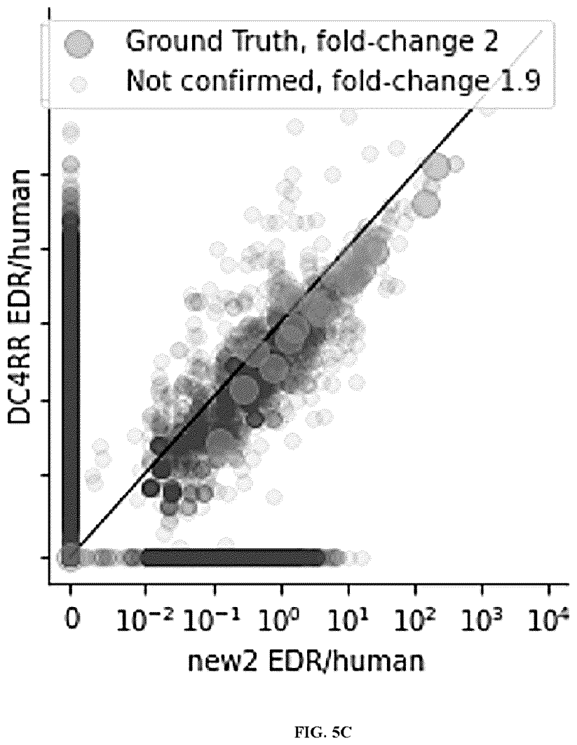

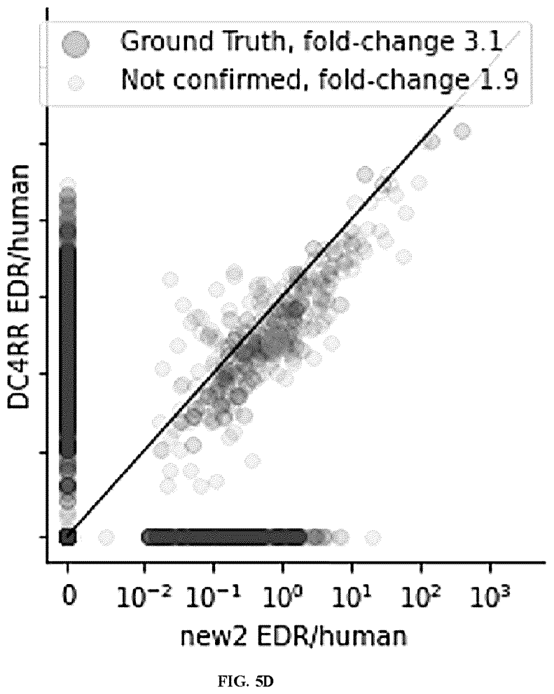

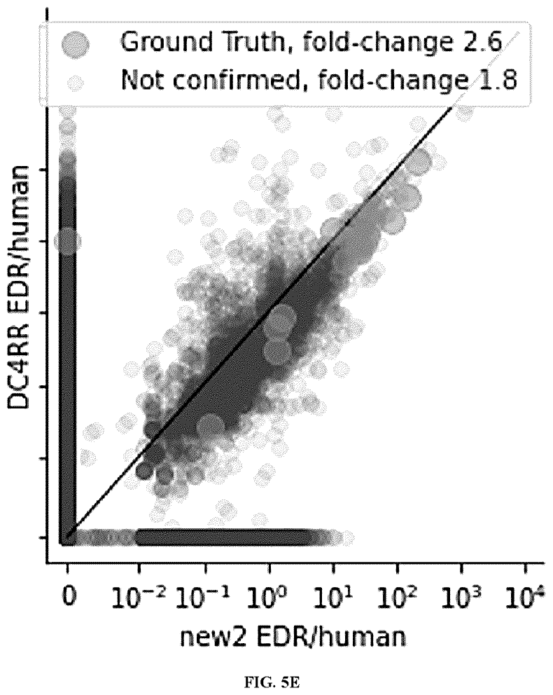

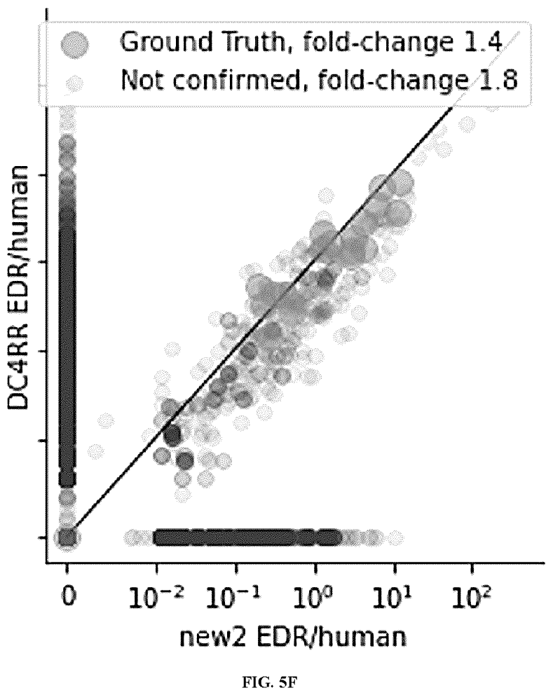

FIG. 5A-5F are results of enrichment of human patients using dummy oligos. FIG. 5A are results with Staphyloccocus aureus in plasma of S. aureus-infected patients. S. aureus shows 2.7×EDR/ddHuman enrichment with a dummy oligo v. without a dummy oligo. Gray dots are SA, black dots are other microbial organisms in the samples. FIG. 5B are results with Aspergillus in plasma of Aspergillus-infected patients. Aspergillus shows no EDR/ddHuman enrichment (or depletion) with a dummy oligo v. without a dummy oligo. Gray dots are Aspergillus, black dots are other microorganisms in the samples. FIG. 5C are results with BRIC in BRIC-patients. BRIC is a set of pathogens often seen in immune compromised patients. BRIC pathogens show 2×EDR/ddHuman enrichment with a dummy oligo v. without a dummy oligo. FIGS. 5A-5F show the enrichment obtained from testing mcfNA in plasma samples of patients who are known to be infected by a microorganism using the method disclosed herein with dummy oligos. The results show a 2.7-fold increase for plasma from S. aureus-infected patients (FIG. 5A) and a 2-fold increase for immune compromised patients (FIG. 5C). Improvements were not initially found in plasma from patients with aspergillus infections (FIG. 5B). Improvement in sensitivity was seen generally in plasma from viral-infected patients (3.1-fold improvement, FIG. 5D), plasma from bacteria-infected patients (2.6-fold improvement, FIG. 5E), and in plasma from eukaryote-infected patients (1.4-fold improvement, FIG. 5F).

1.-84. (canceled)

85. A method of preparing a nucleic acid library enriched for microbial nucleic acids relative to human nucleic acids from a sample comprising microbial nucleic acids and human nucleic acids, the method comprising:
(a) providing the sample, wherein a first population of nucleic acids comprising the human nucleic acids comprises 5′-phosphorylated termini and a second population of nucleic acids comprising the microbial nucleic acids comprises 5′ termini lacking a phosphate;
(b) selectively ligating a blocking oligonucleotide to the 5′-phosphorylated termini of the first population to form blocked first-population nucleic acids, wherein the blocking oligonucleotide inhibits phosphorylation and/or ligation at a 5′ terminus of the blocked first-population nucleic acids;
(c) phosphorylating the 5′ termini of the second population using a polynucleotide kinase; and
(d) attaching at least one adapter to nucleic acids of the phosphorylated second population to produce the nucleic acid library,
thereby enriching the microbial nucleic acids relative to the human nucleic acids in the nucleic acid library.

86. The method of claim 85, wherein the sample is a biological fluid sample.

87. The method of claim 86, wherein the biological fluid sample is selected from the group consisting of blood, serum, plasma, bronchial lavage, synovial fluid, bronchoalveolar lavage, and cerebrospinal fluid.

88. The method of claim 87, wherein the biological fluid sample is plasma.

89. The method of claim 85, wherein the sample is from a human subject.

90. The method of claim 85, wherein the microbial nucleic acids are selected from the group consisting of bacterial nucleic acids, fungal nucleic acids, parasite nucleic acids, protozoa nucleic acids, and viral nucleic acids.

91. The method of claim 85, wherein the first population and/or the second population comprises cell-free DNA.

92. The method of claim 85, wherein the first population comprises human cell-free DNA and the second population comprises microbial cell-free DNA.

93. The method of claim 85, wherein the blocking oligonucleotide comprises an identifying tag sequence.

94. The method of claim 85, wherein the blocking oligonucleotide generates a 5′ terminus that is (i) resistant to phosphorylation by the polynucleotide kinase and/or (ii) resistant to ligation to an adapter.

95. The method of claim 85, wherein the blocking oligonucleotide comprises a splint oligonucleotide.

96. The method of claim 95, wherein the splint oligonucleotide comprises a double-stranded blocking adapter region and a single-stranded region.

97. The method of claim 96, wherein a splint region of the splint oligonucleotide comprises a first strand of the double-stranded blocking adapter region annealed to the single-stranded region.

98. The method of claim 96, wherein the single-stranded region comprises random nucleotides.

99. The method of claim 95, wherein the splint oligonucleotide comprises at least one uracil base.

100. The method of claim 99, further comprising cleaving the at least one uracil base using a uracil-DNA glycosylase reaction.

101. The method of claim 85, wherein the blocking oligonucleotide is coupled to a bead-binding moiety, and the method further comprises removing the blocked first-population nucleic acids by physical separation prior to step (d).

102. The method of claim 85, wherein the polynucleotide kinase is T4 polynucleotide kinase.

103. The method of claim 85, wherein attaching the at least one adapter in step (d) comprises ligating a 5′ adapter to the phosphorylated 5′ termini of the second population.

104. The method of claim 103, wherein ligating the 5′ adapter is performed using a ligase selected from the group consisting of T4 DNA ligase, SplintR ligase, PBCV-1 DNA ligase, and Chlorella virus DNA ligase.

105. The method of claim 85, further comprising attaching a 3′ adapter to 3′ termini of nucleic acids in the sample.

106. The method of claim 85, wherein attaching the at least one adapter comprises ligation, primer extension, template switching, or a combination thereof.

107. The method of claim 85, further comprising performing an assay on the nucleic acid library to detect and/or quantify at least one microbial nucleic acid.

108. The method of claim 107, wherein the assay comprises a PCR assay.

109. The method of claim 107, wherein the assay comprises a sequencing assay.

110. The method of claim 109, wherein the sequencing assay comprises a next generation sequencing assay.

111. A kit for preparing a nucleic acid library enriched for microbial nucleic acids relative to human nucleic acids from a sample, the kit comprising:
(a) a 5′ blocking splint oligonucleotide comprising (i) a double-stranded blocking adapter region and (ii) a single-stranded region, wherein the 5′ blocking splint oligonucleotide comprises at least one uracil base and has a 5′ terminus that is not phosphorylated by a polynucleotide kinase under phosphorylation conditions;
(b) a polynucleotide kinase;
(c) a 5′ splint oligonucleotide configured for ligating a 5′ adapter to a 5′-phosphorylated nucleic acid;
(d) a ligase; and
(e) a uracil-DNA glycosylase reagent.

112. A system configured to prepare a nucleic acid library enriched for microbial nucleic acids relative to human nucleic acids, the system comprising:
a processor; and
a non-transitory memory storing instructions that, when executed by the processor, cause the system to perform the method of claim 85.
